(19) United States
(12) Patent Application Publication
(10) Pub. No.: US 2022/0241167 A1
(43) Pub. Date: Aug. 4, 2022

(54) PRODUCT FOR TREATING KERATINOUS FIBERS, CONTAINING SILANES OF SPECIFIC FORMULAE

(71) Applicant: Henkel AG & Co. KGaA, Duesseldorf (DE)

(72) Inventors: Torsten LECHNER, Langenfeld (DE); Juergen SCHOEPGENS, Schwalmtal (DE)

(73) Assignee: Henkel AG & Co. KGaA, Duesseldorf (DE)

(21) Appl. No.: 17/611,526

(22) PCT Filed: Feb. 27, 2020
(86) PCT No.: PCT/EP2020/055150

§ 371 Date: Nov. 15, 2021

(30) Foreign Application Priority Data
May 15, 2019 (DE) 10 2019 207 060.4

(51) Int. Cl. A61K 8/41; A61Q 5/06

(52) U.S. Cl. CPC A61K 8/41; A61K 2800/432; A61Q 5/065

(57) ABSTRACT

A cosmetic composition for the treatment of keratinous material, in particular keratinous fibers, includes
(a) at least one silane of formula (I)

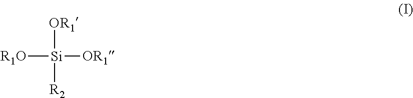

(b) at least one silane comprising at least one structural unit of formula (II),

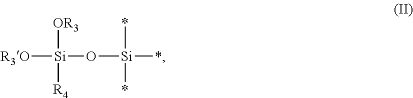

(c) at least one silane comprising at least one structural unit of formula (III), and

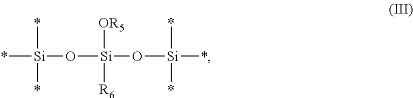

(d) at least one silane comprising at least one structural unit of formula (IV),

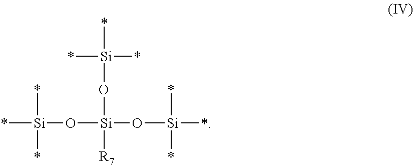

### CROSS-REFERENCE TO RELATED APPLICATION

This application is a U.S. National-Stage entry under 35 U.S.C. § 371 based on International Application No. PCT/EP2020/055150, filed Feb. 27, 2020, which was published under PCT Article 21(2) and which claims priority to German Application No. 102019207060.4, filed May 15, 2019, which are all hereby incorporated in their entirety by reference.

### TECHNICAL FIELD

The present application is in the field of cosmetics and relates to a cosmetic composition comprising a mixture of a monomeric silane compound (a) of formula (I), a singly crosslinked silane (b) having at least one structural unit of formula (II), a doubly crosslinked silane (c) having at least one structural unit of formula (III) and a fully crosslinked silane (d) having at least one structural unit of formula (IV).

### BACKGROUND

A second object of the present disclosure is a multicomponent packaging unit (kit-of-parts) for coloring keratinous material, which comprises, separately packaged in two packaging units, the cosmetic compositions (A) and (B), the composition (A) being a composition of the first object of the disclosure and the composition (B) comprising at least one coloring compound.

The change in shape and color of keratin fibers, especially hair, is an important area of modern cosmetics. To change the hair color, the expert knows various coloring systems depending on coloring requirements. Oxidation dyes are usually used for permanent, intensive dyeing's with good fastness properties and good grey coverage. Such dyes usually contain oxidation dye precursors, so-called developer components and coupler components, which form the actual dyes with one another under the influence of oxidizing agents, such as hydrogen peroxide. Oxidation dyes are exemplified by very long-lasting dyeing results.

When direct dyes are used, ready-made dyes diffuse from the colorant into the hair fiber. Compared to oxidative hair dyeing, the dyeing's obtained with direct dyes have a shorter shelf life and quicker wash ability. Dyeing with direct dyes usually remain on the hair for a period of between 5 and 20 washes.

The use of color pigments is known for short-term color changes on the hair and/or skin. Color pigments are generally understood to be insoluble, coloring substances. These are present undissolved in the dye formulation in the form of small particles and are only deposited from the outside on the hair fibers and/or the skin surface. Therefore, they can usually be removed again without residue by a few washes with detergents comprising surfactants. Various products of this type are available on the market under the name hair mascara.

If the user wants particularly long-lasting dyeing's, the use of oxidative dyes has so far been his only option. However, despite numerous optimization attempts, an unpleasant ammonia or amine odor cannot be completely avoided in oxidative hair dyeing. The hair damage still associated with the use of oxidative dyes also has a negative effect on the user's hair.

EP 2168633 B1 deals with the task of producing long-lasting hair colorations using pigments. The paper teaches that when a combination of pigment, organic silicon compound, hydrophobic polymer and a solvent is used on hair, it is possible to produce colorations that are particularly resistant to shampooing.

The organic silicon compounds used in EP 2168633 B1 are reactive compounds from the class of alkoxy silanes. These alkoxy silanes hydrolyze at high rates in the presence of water and form hydrolysis products and/or condensation products, depending on the amounts of alkoxy silane and water used in each case. The influence of the amount of water used in this reaction on the properties of the hydrolysis or condensation product are described, for example, in WO 2013068979 A2.

When these hydrolysis or condensation products are applied to keratinous material, a film or coating is formed on the keratinous material, which completely envelops the keratinous material and, in this way, strongly influences the properties of the keratinous material. Possible areas of application include permanent styling or permanent shape modification of keratin fibers. In this process, the keratin fibers are mechanically shaped into the desired form and then fixed in this form by forming the coating described above. Another particularly suitable application is the coloring of keratin material; in this application, the coating or film is produced in the presence of a coloring compound, for example a pigment. The film colored by the pigment remains on the keratin material or keratin fibers and results in surprisingly wash-resistant colorations.

The great advantage of the alkoxy silane-based dyeing principle is that the high reactivity of this class of compounds enables very fast coating. This means that extremely good coloring results can be achieved after very short application periods of just a few minutes. In addition to these advantages, however, the high reactivity of alkoxy silanes also has some disadvantages. Thus, even minor changes in production and application conditions, such as changes in humidity and/or temperature, can lead to sharp fluctuations in product performance. Most importantly, the work leading to this disclosure has shown that the alkoxy silanes are extremely sensitive to the conditions encountered during the manufacture and storage of the keratin treatment compositions.

If these manufacturing conditions deviate only slightly from their optimal range of values, this can lead to partial or even complete loss of product performance. In this context, it has also been found that the conditions prevailing during storage can also have a very strong influence on the dyeing performance of an alkoxy silane-comprising colorant.

On contact with water, the alkoxy silanes can undergo complex hydrolysis and condensation reactions that lead to mixtures of monomeric, dimeric, and oligomeric compounds in equilibrium with each other. If the alkoxysilanes are compounds that contain several hydrolysable alkoxy groups, each alkoxy-silane can also undergo several condensation reactions. Depending on the number of condensations per alkoxysilane molecule, the formation of linear condensates as well as the formation of cross-linked, three-dimensional networks is possible.

The reaction mechanisms and reaction equilibria of alkoxy-silanes such as 3-aminopropyltriethoxysilane that occur in these condensations have been studied, for example, in Journal of Organometallic Chemistry 625 (2001), 208-216. Various technical application tests have now shown that the performance of a keratin treatment agent can depend significantly on which oligomers with which degree of cross-linking are used in the keratin treatment agent.

### BRIEF SUMMARY

This disclosure provides a cosmetic composition for the treatment of keratinous material, the composition comprising:

(a) at least one silane of formula (I)

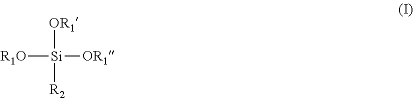

where
R1, R1′, R1″ independently are a hydrogen atom or a C₁-C₆ alkyl group,
R2 is a C₁-C₈ alkyl group or an amino-C₁-C₈ alkyl group, and
(b) at least one silane comprising at least one structural unit of formula (II),

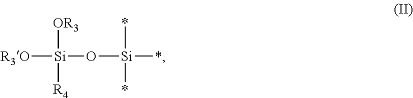

where
R3, R3′ independently are a hydrogen atom or a C₁-C₆ alkyl group, and
R4 is a C₁-C₈ alkyl group or an amino-C₁-C₈ alkyl group, and
(c) at least one silane comprising at least one structural unit of formula (III),

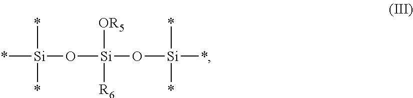

where
R5 is a hydrogen atom or a C₁-C₆ alkyl group, and
R6 is a C₁-C₈ alkyl group or an amino-C₁-C₈ alkyl group, and
(d) at least one silane comprising at least one structural unit of formula (IV),

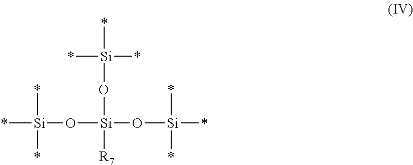

where
R7 is a C₁-C₈ alkyl group or an amino-C₁-C₈ alkyl group.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

It was the task of the present application to find a composition for the treatment of keratin material which comprises the silane oligomers or comprises silane condensates in an optimal mixture and composition. The aim was to hydrolyze and condense the alkoxy silanes used to prepare the agent in a targeted manner so that compositions with optimum application properties could be obtained. In particular, the agents prepared by this method should have improved dyeing performance, i.e., when used in a dyeing process, dyeing's with higher color intensity and improved fastness properties, especially improved wash fastness and improved rub fastness, should be obtained.

Surprisingly, it has now been found that the task can be excellently solved if a composition is used for the treatment of the keratin material which comprises a mixture of monomeric silanes (a) of a formula (I), dimeric or linear silane condensates (b) and (c) with structural units of the formulae (II) and (II) as well as crosslinked silane condensates (d) with structural units of the formula (IV).

A first object of the present disclosure is a cosmetic composition for treating keratinous material, in particular keratinous fibers, comprising.

(a) at least one silane of formula (I)

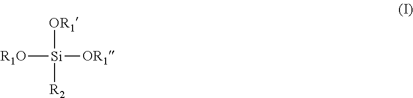

where

R1, R1′, R1″ independently represent a hydrogen atom or a C₁-C₆ alkyl group,
R2 is a C₁-C₈ alkyl group or an amino-C₁-C₈ alkyl group, and
(b) at least one silane comprising at least one structural unit of formula (II),

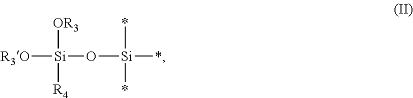

where
R3, R3′ independently represent a hydrogen atom or a C₁-C₆ alkyl group, and
R4 is a C₁-C₈ alkyl group or an amino-C₁-C₈ alkyl group, and
(c) at least one silane comprising at least one structural unit of formula (III),

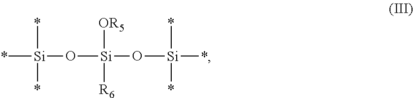

where

R5 represents a hydrogen atom or a C₁-C₆ alkyl group, and
R6 is a C₁-C₈ alkyl group or an amino-C₁-C₈ alkyl group, and
(d) at least one silane comprising at least one structural unit of formula (IV),

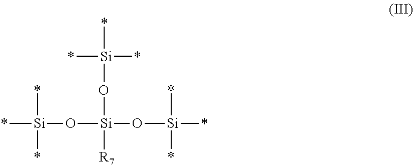

where

R7 is a C₁-C₈ alkyl group or an amino-C₁-C₈ alkyl group.

It has been shown that hair treatment compositions comprising the above composition, when used in a dyeing process, resulted in very intense and uniform colorations with very good rub fastness and wash fastness.

### Agent for the Treatment of Keratinous Material

Keratinous material includes hair, skin, nails (such as fingernails and/or toenails). Wool, furs, and feathers also fall under the definition of keratinous material.

Preferably, keratinous material is understood to be human hair, human skin, and human nails, especially fingernails and toenails. Keratinous material is understood to be human hair.

Agents for treating keratinous material are understood to mean, for example, features for coloring the keratinous material, features for reshaping or shaping keratinous material, in particular keratinous fibers, or also features for conditioning or caring for the keratinous material. The cosmetic compositions as contemplated herein show particularly good suitability for coloring keratinous material, for coloring keratinous fibers, which are especially preferably human hair.

The term “coloring agent” is used in the context of the present disclosure to refer to a coloring of the keratin material, of the hair, caused using coloring compounds, such as thermochromic and photochromic dyes, pigments, mica, direct dyes and/or oxidation dyes. In this staining process, the colorant compounds are deposited in a particularly homogeneous and smooth film on the surface of the keratin material or diffuse into the keratin fiber. The film forms in situ by oligomerization or polymerization of the organic silicon compound(s), and by the interaction of the color-imparting compound and organic silicon compound and optionally other ingredients, such as a film-forming hydrophilic polymer.

### Silanes (a) of the Formula (I)

A typical feature of the compositions as contemplated herein is their content of at least one silane (a) of the formula (I),

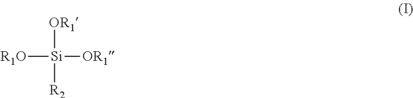

where
R1, R1′, R1″ independently represent a hydrogen atom or a C₁-C₆ alkyl group,
R2 is a C₁-C₈ alkyl group or an amino-C₁-C₈ alkyl group.

The silanes of formula (I) are monomeric silanes. If at least one of the radicals R1, R1′ and R1″ stands for a C₁-C₆ alkyl group, these compounds can also be called C₁-C₆ alkoxysilanes. Silanes in which the radicals R1, R1′ and R1″ stand for a hydrogen can also be called silanols.

The C₁-C₆ alkoxy silanes of formula (I) are each highly reactive compounds that undergo a hydrolysis reaction in the presence of water. This hydrolysis reaction is exothermic and starts when the silanes (I) meet water. The reaction product is the corresponding hydroxy silane in which at least the corresponding radical R1, R1′ and/or represents a hydrogen atom. The hydroxy silane may alternatively be referred to as silanol.

The organic C₁-C₆ alkoxy silane(s) are organic, non-polymeric silicon compounds.

Organic silicon compounds, alternatively called organosilicon compounds, are compounds which either have a direct silicon-carbon bond (Si—C) or in which the carbon is bonded to the silicon atom via an oxygen, nitrogen, or sulfur atom.

According to IUPAC rules, the term silane chemical compounds based on a silicon skeleton and hydrogen. In organic silanes, the hydrogen atoms are completely or partially replaced by organic groups such as (substituted) alkyl groups and/or alkoxy groups. A typical feature of the C₁-C₆ alkoxy silanes of the disclosure is that at least one C₁-C₆ alkoxy group is directly bonded to the silicon atom.

The substituents R1, R1′ R1″ and R2 in the compounds of formula (I) are explained below by way of example:

Examples of a C₁-C₆ alkyl group are the groups methyl, ethyl, propyl, isopropyl, n-butyl, s-butyl and t-butyl, n-pentyl and n-hexyl. Propyl, ethyl, and methyl are preferred alkyl radicals. Examples of a C₁-C₈ alkyl group are, in addition to the alkyl groups, an n-hexyl group and an n-octyl group.
Examples of an R2 for an amino-C₁-C₈ alkyl group are the aminomethyl group, the 2-aminoethyl group, the 3-aminopropyl group, the 4-aminobutyl group, the 5-aminopentyl group, the 6-aminohexyl group. The 3-aminopropyl group is particularly preferred.

The R1 radical in the silanes of formula (I) represents a hydrogen atom or a C₁-C₆ alkyl group. Very preferably, the radical R1 represents a hydrogen atom, a methyl group, or an ethyl group.

In the silanes of formula (I), the radical R2 represents a C₁-C₈ alkyl group or an amino-C₁-C₈ alkyl group. Very preferably, the radical R2 represents a methyl group, an ethyl group, an n-hexyl group, an n-octyl group and a 3-aminopropyl group.

Keratin treatment agents with particularly good properties could be prepared if the composition as contemplated herein included at least one silane (a) of formula (I) selected from the group of:

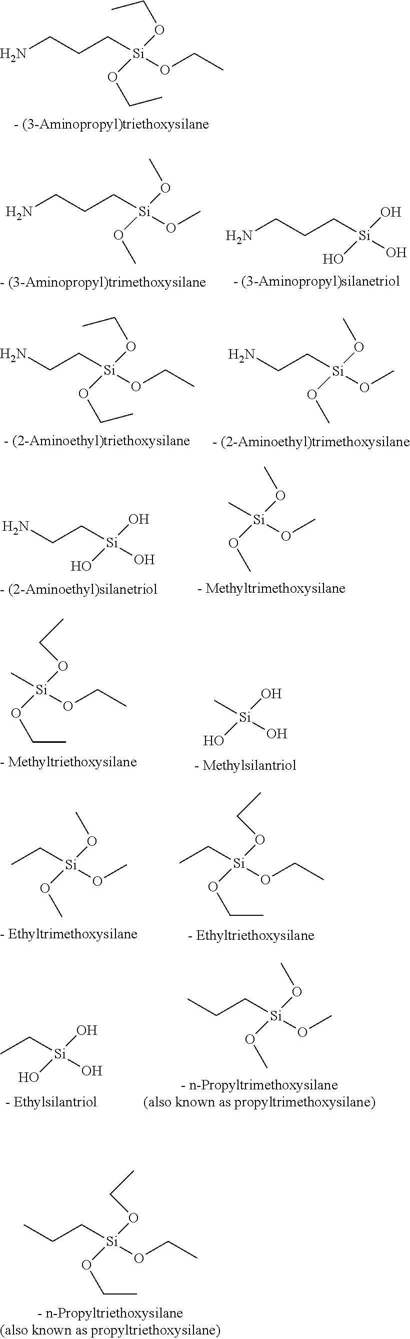

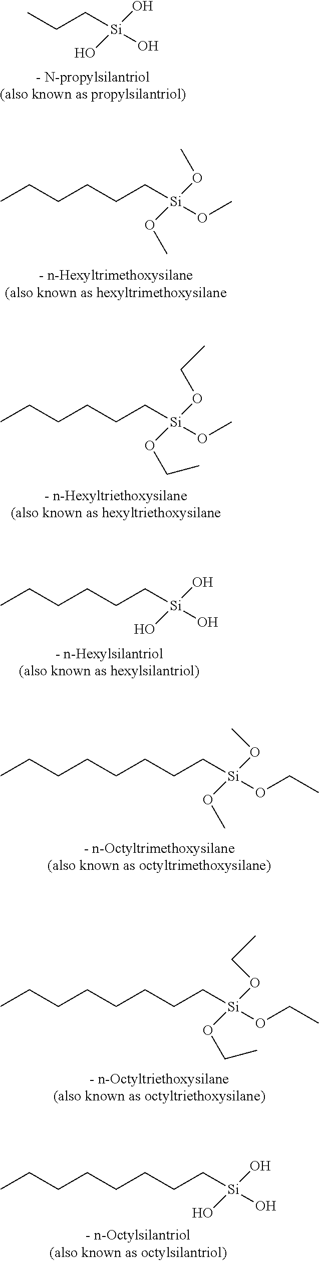

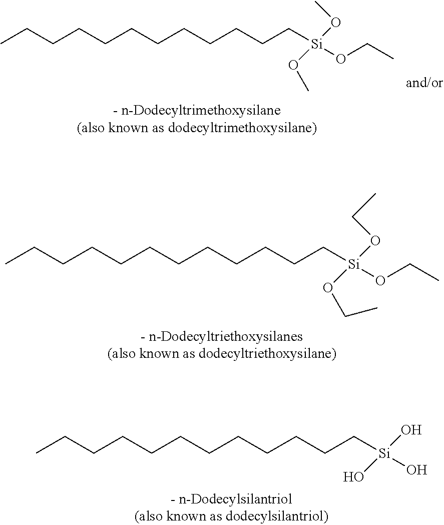

The presence of very specific silanes of formula (I) has proven to be particularly advantageous regarding achieving good application properties. When the compositions as contemplated herein are used as a coloring agent, particularly intensively colored keratin materials could be obtained when the composition is included at least one silane (a) of the formula (Ia)

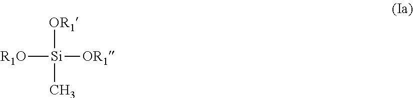

where
R1, R1′, R1″ independently represent a hydrogen atom or a C₁-C₆ alkyl group.

In a very particularly preferred embodiment, a composition as contemplated herein is wherein it comprises at least one silane (a) of the formula (Ia)

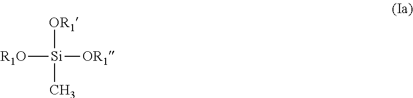

where
R1, R1′, R1″ independently of one another represent a hydrogen atom or a C1-C8 alkyl group.
R1, R1′, R1″ independently represent a hydrogen atom or a C₁-C₈ alkyl group. Very preferably, R1, R1′ and R1″ independently represent a hydrogen atom, a methyl group or an ethyl group.

When the composition as contemplated herein was used in a dyeing composition, it was possible to obtain very intensively colored keratin materials when the composition included at least one silane (a) of the formula (Ib)

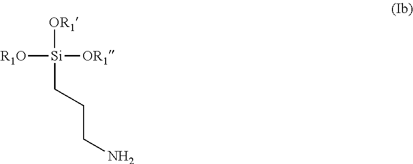

where
R1, R1′, R1″ independently represent a hydrogen atom or a C₁-C₈ alkyl group.

In the context of a further very particularly preferred embodiment, a composition as contemplated herein is wherein it comprises at least one silane (a) of the formula (Ib)

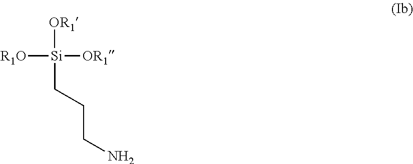

where

R1, R1′, R1″ independently represent a hydrogen atom or a C₁-C₆ alkyl group.

R1, R1′, R1″ independently represent a hydrogen atom or a C₁-C₆ alkyl group. Very preferably, R1, R1′ and R1″ independently represent a hydrogen atom, a methyl group or an ethyl group.

The best application properties were observed with compositions comprising at least one silane of formula (Ia) as well as one silane of formula (Ib).

In an explicitly very particularly preferred embodiment, a composition as contemplated herein is wherein it comprises at least one silane (a) of the formula (Ia) and at least one silane of the formula (Ib)

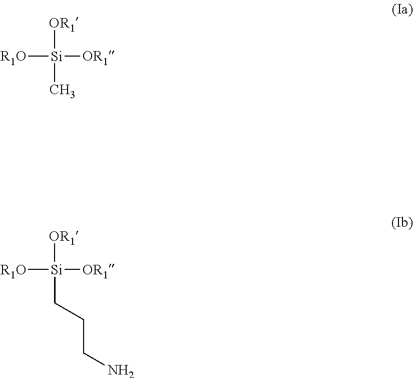

where
the radicals R1, R1′, R1″ in the formula (Ia) can be chosen independently of the radicals R1, R1′, R1″ in the formula (Ib) and independently of one another represent a hydrogen atom or a C₁-C₆ alkyl group, particularly preferably a hydrogen atom, a methyl group, or an ethyl group.

Very particularly preferred compositions as contemplated herein for treating keratin fibers can be prepared, for example, by mixing one or more C₁-C₆ alkoxy silanes of formula (I) with water.

In one embodiment, a cosmetic composition for treating keratinous material, in particular keratinous fibers, comprising a product obtained by mixing (3-aminopropyl)triethoxysilane with methyltrimethoxysilane and water is particularly preferred.

In the context of one embodiment, very particularly preferred is a cosmetic composition for treating keratinous material, in particular keratinous fibers, comprising a product obtained by mixing (3-aminopropyl)triethoxysilane with ethyltriethoxysilane and water.

In the context of one embodiment, very particularly preferred is a cosmetic composition for treating keratinous material, in particular keratinous fibers, comprising a product obtained by mixing (3-aminopropyl)triethoxysilane with methyltriethoxysilane and water.

In the context of one embodiment, very particularly preferred is a cosmetic composition for treating keratinous material, in particular keratinous fibers, comprising a product obtained by mixing (3-aminopropyl)triethoxysilane with propyltriethoxysilane and water.

In the context of one embodiment, very particularly preferred is a cosmetic composition for treating keratinous material, in particular keratinous fibers, comprising a product obtained by mixing (3-aminopropyl)triethoxysilane with hexyltriethoxysilane and water.

In the context of one embodiment, very particularly preferred is a cosmetic composition for treating keratinous material, in particular keratinous fibers, comprising a product obtained by mixing (3-aminopropyl)trimethoxysilane with hexyltriethoxysilane and water.

In the context of one embodiment, very particularly preferred is a cosmetic composition for treating keratinous material, in particular keratinous fibers, comprising a product obtained by mixing (3-aminopropyl)triethoxysilane with octyltriethoxysilane and water.

In the context of one embodiment, very particularly preferred is a cosmetic composition for treating keratinous material, in particular keratinous fibers, comprising a product obtained by mixing (3-aminopropyl)triethoxysilane with octyltrimethoxysilane and water.

Since the silanes of the structural groups can each react with water during hydrolysis and with each other during a subsequent condensation, the reactions taking place in the composition are very complex, and mixtures of monomeric and oligomeric silane condensates are formed with the mixing. It is believed that the following reactions are initiated in these mixtures:

Hydrolysis of C₁-C₆ alkoxy silane of formula (I) with water (reaction scheme using 3-aminopropyltriethoxysilane as an example):

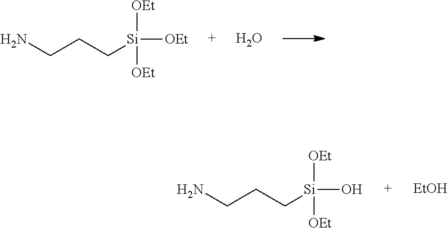 [SMILES: "EtOH" ; CCO[Si](CCCN)(OCC)OCC ; O ; CCO[Si](O)(CCCN)OCC]

Depending on the amount of water used, the hydrolysis reaction can also take place several times per C₁-C₆ alkoxy silane used:

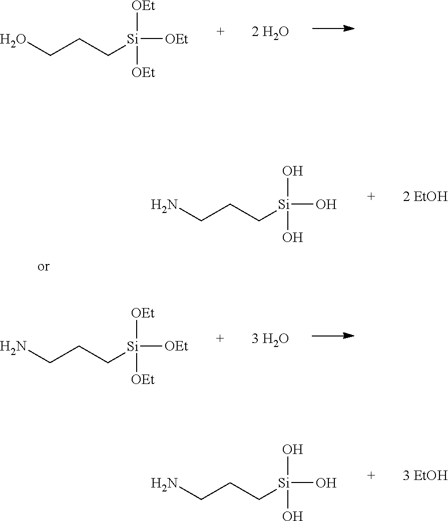 [SMILES: CCO[Si](CCCO)(OCC)OCC ; NCCC[Si](O)(O)O ; CCO[Si](CCCN)(OCC)OCC ; NCCC[Si](O)(O)O]

Hydrolysis of C₁-C₆ alkoxy silane of formula (I) with water (reaction scheme using methyltrimethoxysilane as an example):

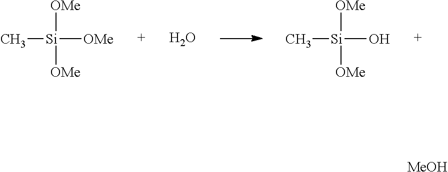

Depending on the amount of water used, the hydrolysis reaction can also take place several times per C₁-C₆ alkoxy silane used:

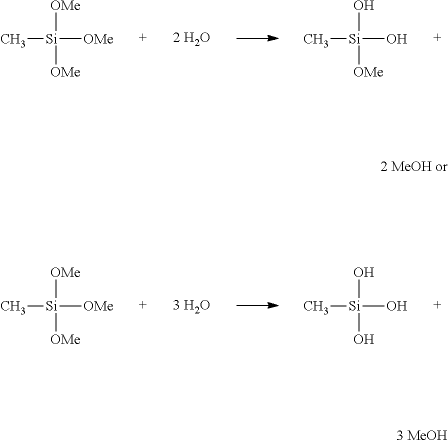

The silanes of formula (I) are the C₁-C₆ alkoxysilanes described above or their hydrolysis products.

It has been found to be particularly preferable for a certain proportion of these silanes (I) to remain in the composition in their monomeric form, and for the larger proportion of the silanes to react further to form oligomeric condensates.

With compositions as contemplated herein which—based on the total molar amount of all silicon compounds used in the composition—included one or more silanes (a) of formula (I) in a total molar proportion of about 0.3 to about 12.0 mol %, preferably about 0.6 to about 10.0 mol %, further preferably about 0.8 to about 7.0 mol % and very particularly preferably about 1.4 to about 6.0 mol %, very particularly good and intensive color results were obtained.

In the context of a further embodiment, a composition comprising—based on the total molar amount of all silicon compounds used in the composition—is very particularly preferred

(a) one or more silanes of formula (I) in a total mole fraction of from about 0.3 to about 12.0 mol %, preferably from about 0.6 to about 10.0 mol %, more preferably from about 0.8 to about 7.0 mol % and most preferably from about 1.4 to about 6.0 mol %.

It is further particularly preferred if the composition as contemplated herein comprises the silanes of formula (Ia) in certain molar proportions. With compositions as contemplated herein which contain—based on the total molar amount of all silicon compounds used in the composition—(a) one or more silanes of the formula (Ia) in a total molar proportion of about 0.2 to about 7.0 mol %, preferably about 0.4 to about 6.0 mol %, further preferably about 0.5 to about 5.0 mol % and very particularly preferably about 1.0 to about 4.0 mol %, very particularly good and intensive color results were obtained.

In the context of a further embodiment, a composition comprising—based on the total molar amount of all silicon compounds used in the composition—(a) one or more silanes of the formula (Ia) in a total molar proportion of from about 0.2 to about 7.0 mol %, preferably from about 0.4 to about 6.0 mol %, further preferably from about 0.5 to about 5.0 mol % and very particularly preferably from about 1.0 to about 4.0 mol % is very particularly preferred.

It is further particularly preferred if the composition as contemplated herein comprises the silanes of formula (Ib) in certain molar proportions. With compositions as contemplated herein which contain—based on the total molar amount of all silicon compounds used in the composition—(a) one or more silanes of the formula (Ib) in a total molar proportion of about 0.1 to about 5.0 mol %, preferably about 0.2 to about 4.0 mol %, further preferably about 0.3 to about 2.0 mol % and very particularly preferably about 0.4 to about 2.0 mol %, very particularly good and intensive color results were obtained.

In the context of a further embodiment, a composition comprising—based on the total molar amount of all silicon compounds used in the composition—(a) one or more silanes of the formula (Ib) in a total molar proportion of from about 0.1 to about 5.0 mol %, preferably from about 0.2 to about 4.0 mol %, further preferably from about 0.3 to about 2.0 mol % and very particularly preferably from about 0.4 to about 2.0 mol % is very particularly preferred.

The determination of the amount of silanes (a) of formula (I) included in the composition as contemplated herein is particularly preferably carried out by quantitative 29 silicon NMR spectroscopy.

The content of silanes (b), (c) and (d) can also be determined in an analogous manner. For the measurement, for example, the procedure described below can be used.

### Quantitative 29Si NMR Spectroscopy

Use of NMR sample tubes

Device: Agilent, 600 MHz

29Si-NMR spectra were recorded in chloroform from each of the compositions. Measurements were taken on the day of production, after 7 days and after 14 days.

Standard: TMS (tetramethylsilane)

Relaxation accelerator: Chromium(III) acetylacetonate

By using the relaxation accelerator, the integrals of the individual signals became comparable with each other. The sum over all integrals was set equal to 100 mol %.
For the quantitative determination, the area of each individual signal was related to the total sum over all integrals.
The measurement of the spectra was carried out according to the procedure described in Journal of Organometallic Chemistry 625 (2001), 208-216.

### Silanes (b) Comprising at Least One Structural Unit of Formula (II)

A further typical feature for the compositions as contemplated herein is their content of at least one silane (b) which comprises at least one structural unit of the formula (II)

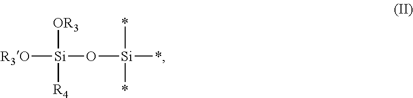

where
R3, R3′ independently represent a hydrogen atom or a C₁-C₆ alkyl group, and
R4 is a C₁-C₈ alkyl group or an amino-C₁-C₈ alkyl group.

The silanes (b) have at least one structural unit of the formula (II). The structural units of formula (II) are simply cross-linked silanes obtained by the further condensation of the monomeric silanes of formula (I). In this condensation, a monomeric silane (i.e., the structural subunit in formula (II) bearing the radicals R3 and R4) reacts with at least one other silane with elimination of water or alcohol.

In the structural unit of formula (II), R3, R3′ independently represent a hydrogen atom or a C₁-C₆ alkyl group. Very preferably, the radicals R3 and R3′ independently of one another represent a hydrogen atom, a methyl group or an ethyl group.

In the structural unit of formula (II), the radical R4 represents a C₁-C₈ alkyl group or an amino-C₁-C₈ alkyl group. Very preferably, the radical R4 represents a methyl group, an ethyl group, an n-hexyl group, an n-octyl group or a 3-aminopropyl group.

The bonds in the structural unit of formula (II), which start from the silicon atom and are marked with an asterisk, represent the further free bond valences of this silicon atom, i.e., this Si atom has three further bonds, which preferably go to a further carbon or to an oxygen atom.

The structural unit of formula (II) is thus exemplified by the fact that it comprises a single cross-linked silicon atom which has a further bond to a second silicon atom via the oxygen atom.

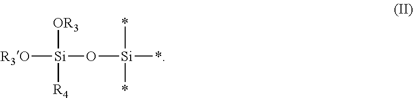

In a particularly preferred embodiment, the silanes (b), which may be comprising at least one structural unit of the formula (II), for example, are dimeric compounds which can be formed via the following reactions:

Possible condensation reactions shown using the mixture of (3-aminopropyl)triethoxysilane and methyltrimethoxysilane:

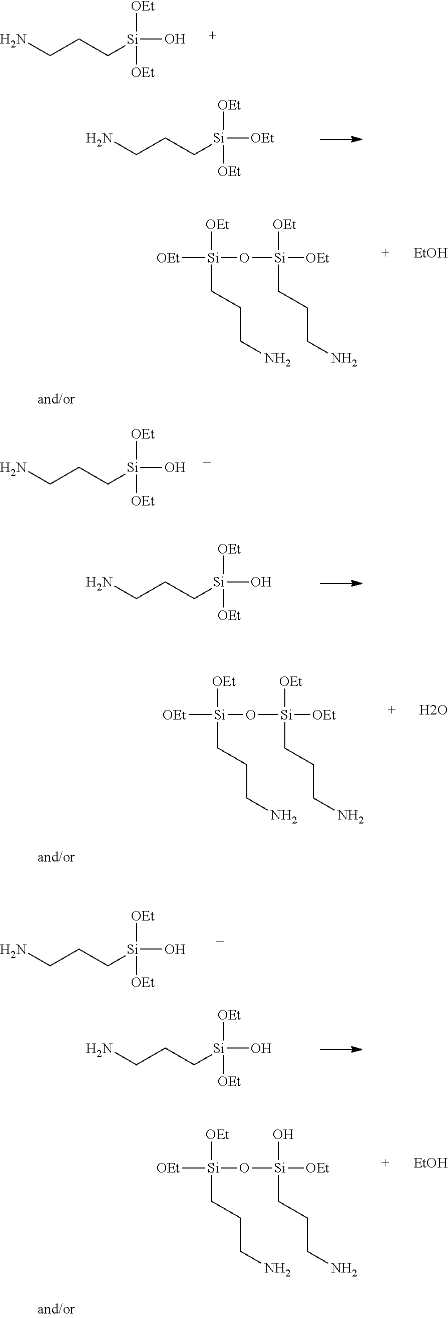

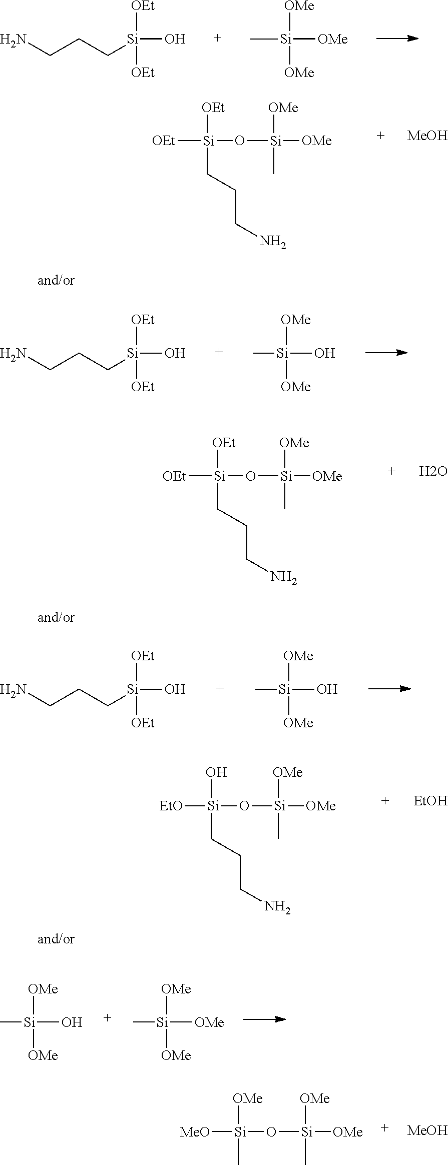

If the silanes (b) with at least one structural unit of formula (II) are the dimers described above, the silanes (b) are structurally different from the silanes of groups (c) and (d).

Each of the previously drawn dimeric silanes (b) comprises two structural units of formula (II).

In another particularly preferred embodiment, the silanes (b) comprising at least one structural unit of formula (II) may also be linear silane oligomers in which the structural units of formula (II) represent the end groups of the linear oligomer.

Possible condensation reactions shown using the mixture of (3-aminopropyl)triethoxysilane and methyltrimethoxysilane:

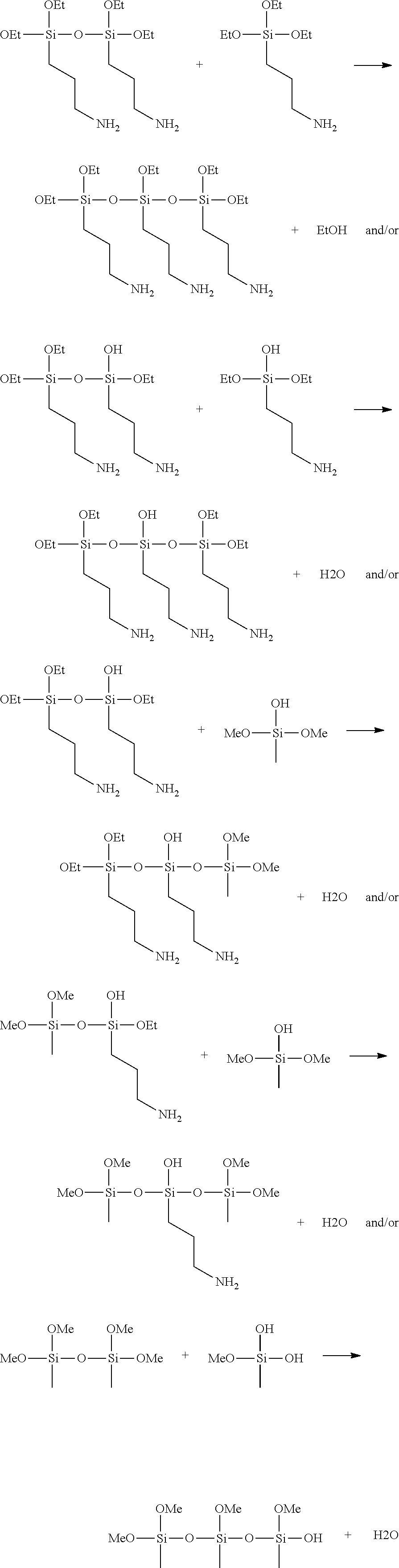

Each of the previously drawn trimeric silanes (b) comprises two structural units of formula (II).

The presence of very specific silanes (b) comprising at least one structural unit of formula (II) has been shown to be particularly advantageous in terms of achieving good application properties. When the compositions as contemplated herein were used as a coloring agent, particularly intensively colored keratin materials could be obtained if the composition included at least one silane (b) comprising at least one structural unit of the formula (IIa),

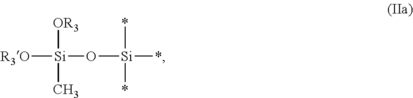

where
R3, R3′ independently represent a hydrogen atom or a C₁-C₆ alkyl group.

In the context of a further very particularly preferred embodiment, a composition as contemplated herein is wherein it comprises at least one silane (b) which comprises at least one structural unit of the formula (IIa),

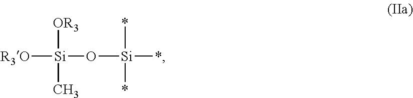

where
R3, R3′ independently represent a hydrogen atom or a C₁-C₆ alkyl group.

The radicals R3 and R3′ independently represent a hydrogen atom or a C₁-C₆ alkyl group. Very preferably, R3 and R3′ independently represent a hydrogen atom, a methyl group or an ethyl group.

When the compositions as contemplated herein were used as a coloring agent, particularly intensively colored keratin materials could also be obtained if the composition included at least one silane (b) comprising at least one structural unit of the formula (IIb),

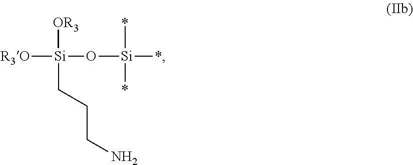

where
R3, R3′ independently represent a hydrogen atom or a C₁-C₆ alkyl group.

In the context of a further very particularly preferred embodiment, a composition as contemplated herein is wherein it comprises at least one silane (b) which comprises at least one structural unit of the formula (IIb),

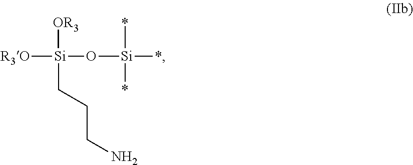

where
R3, R3′ independently represent a hydrogen atom or a C₁-C₆ alkyl group.
R3 and R3′ independently represent a hydrogen atom or a C₁-C₆ alkyl group. Very preferably, R3 and R3′ independently represent a hydrogen atom, a methyl group or an ethyl group.

The best application properties were observed in compositions comprising both at least one silane (b) with at least one structural unit of formula (IIa) and at least one silane (b) with at least one structural unit of formula (IIb).

In an explicitly very particularly preferred embodiment, a composition as contemplated herein is wherein it comprises at least one silane (a) of the formula (Ia) and at least one silane of the formula (Ib)

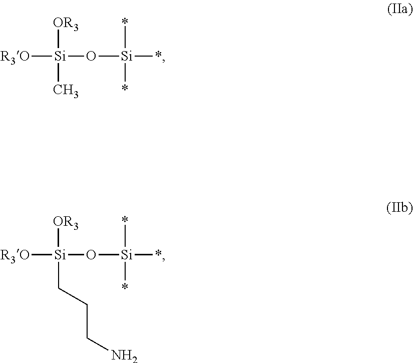

where
the radicals R3, R3′ in the formula (IIa) can be selected independently of the radicals R3, R3′ in the formula (IIb) and independently of one another represent a hydrogen atom or a C₁-C₆ alkyl group, particularly preferably a hydrogen atom, a methyl group, or an ethyl group.

With compositions as contemplated herein which—based on the total molar amount of all silicon compounds used in the composition—included one or more silanes (b) with a total molar proportion of about 12.5 to about 42.0 mol %, preferably of about 14.5 to about 38.0 mol %, further preferably of about 16.0 to about 34.0 mol % and very particularly preferably of about 19.5 to about 30.0 mol % of structural units of formula (II), very particularly good and intensive color results were obtained.

Within the scope of a further embodiment, quite particularly preferred is a A composition comprising—based on the total molar amount of all silicon compounds used in the composition—one or more silanes (b) having a total molar content of from about 12.5 to about 42.0 mol %, preferably from about 14.5 to about 38.0 mol %, further preferably from about 16.0 to about 34.0 mol % and very particularly preferably from about 19.5 to about 30.0 mol % of structural units of the formula (II).

It is further particularly preferred if the composition as contemplated herein comprises the silanes having at least one structural unit of formula (IIa) in certain molar proportions. With compositions as contemplated herein which—based on the total molar amount of all silicon compounds used in the composition—included one or more silanes (b) with a total molar proportion of about 12.0 to about 30.0 mol %, preferably about 14.0 to about 28.0 mol %, further preferably about 14.0 to about 26.0 mol % and very particularly preferably about 16.0 to about 24.0 mol % of structural unit of the formula (IIa), very particularly good and intensive color results were obtained.

Within the scope of a further embodiment, quite particularly preferred is a A composition comprising—based on the total molar amount of all silicon compounds used in the composition—one or more silanes (b) having a total molar content of from about 12.0 to about 30.0 mol %, preferably from about 14.0 to about 28.0 mol %, further preferably from about 14.0 to about 26.0 mol % and very particularly preferably from about 16.0 to about 24.0 mol % of structural units of the formula (IIa).

It is further particularly preferred if the composition as contemplated herein comprises the silanes having at least one structural unit of formula (IIb) in certain molar proportions. With compositions as contemplated herein which—based on the total molar amount of all silicon compounds used in the composition—included one or more silanes (b) with a total molar content of about 0.5 to about 12.0 mol %, preferably of about 1.5 to about 10.0 mol %, further preferably of about 2.5 to about 8.0 mol % and very particularly preferably of about 3.5 to about 6.0 mol % of structural unit of the formula (IIb), very particularly good and intensive color results were obtained.

Within the scope of a further embodiment, quite particularly preferred is a A composition comprising—based on the total molar amount of all silicon compounds used in the composition—one or more silanes (b) having a total molar content of from about 0.5 to about 12.0 mol %, preferably from about 1.5 to about 10.0 mol %, further preferably from about 2.5 to about 8.0 mol % and very particularly preferably from about 3.5 to about 6.0 mol % of structural units of the formula (IIb).

The molar amount of the silanes (b) with a structural unit of the formula (II) included in the composition as contemplated herein is determined, as described above, very preferably by employing quantitative 29-silicon NMR spectroscopy.

### Silanes (c) Comprising at Least One Structural Unit of Formula (III)

A further typical feature of the compositions as contemplated herein is their content of at least one silane (c) which comprises at least one structural unit of the formula (III),

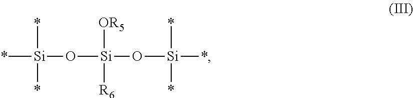

where
R5 represents a hydrogen atom or a C₁-C₆ alkyl group, and
R6 is a C₁-C₈ alkyl group or an amino-C₁-C₈ alkyl group.

The silanes (c) have at least one structural unit of the formula (III). The structural units of formula (III) are doubly cross-linked silanes obtained by the further condensation of the dimeric silanes of formula (II). In this condensation, the dimeric silane reacts with at least one other silane, splitting off water or alcohol.

In the structural unit represented by formula (III), R5 represents a hydrogen atom or a C₁-C₆ alkyl group. Very preferably, the radical R5 represents a hydrogen atom, a methyl group, or an ethyl group.

In the structural unit of formula (III), the radical R6 represents a C₁-C₈ alkyl group or an amino-C₁-C₈ alkyl group. Very preferably, the radical R6 represents a methyl group, an ethyl group, an n-hexyl group, an n-octyl group and a 3-aminopropyl group.

The bonds in the structural unit of formula (III), which start from the two terminal silicon atoms and are marked with an asterisk, represent the further free bond valences of these silicon atoms, i.e., these Si atoms have three further bonds, which preferably go to a further carbon or to an oxygen atom.

The structural unit of the formula (III) is thus exemplified by the fact that it comprises a doubly crosslinked silicon atom which has two further bonds to two silicon atoms via two oxygen atoms

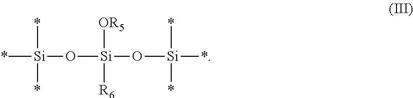

The silanes (c) with at least one structural unit of formula (III) are at least trimeric compounds, i.e., the silanes (c) were obtained by condensation of at least three monomeric C₁-C₆ alkoxysilanes.

Particularly preferably, the silanes (c) are linear oligomers or ring-shaped oligomers, whereby the oligomers can comprise, for example, between about 3 and about 20 structural units of formula (III).

In a particularly preferred embodiment, the silanes (c), which may be comprising at least one structural unit of the formula (III), for example, are linear oligomeric compounds which can be formed via the following reactions:

Possible condensation reactions shown using the mixture of (3-aminopropyl)triethoxysilane and methyltrimethoxysilane:

In linear, trimeric silane condensates, the structural units of formula (III) represent the middle part of the linear trimer. Each of the following trimers comprises a structural unit of formula (III).

Possible condensation reactions to the trimer shown using the mixture of (3-aminopropyl)triethoxysilane and methyltrimethoxysilane:

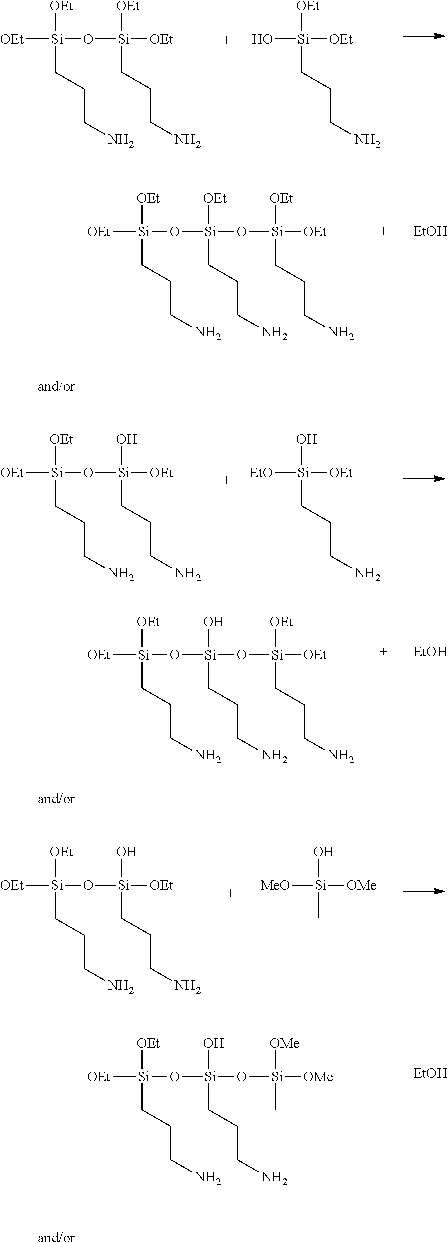

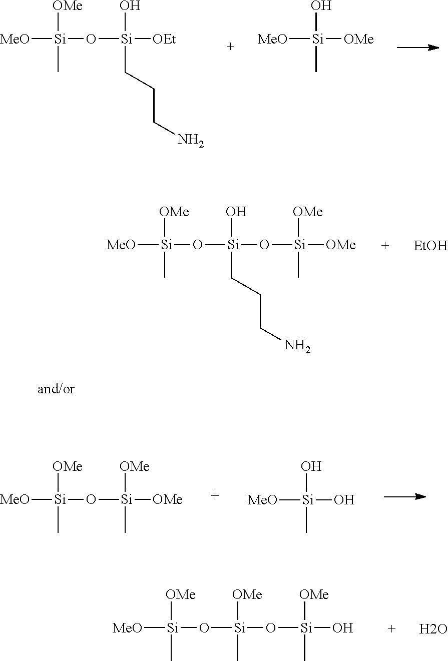

For linear silane condensates with about 4 silane units, the structural units of formula (III) represent the middle part of the linear oligomer. Each of the following silane condensates with about 4 silane units comprises two structural unit of formula (III).

Possible silane condensates with about 4 silane units shown using the mixture (3-aminopropyl)triethoxysilane and methyltrimethoxysilane. The condensation reactions take place in analogy to the reactions already described—only the possible products are shown here as examples:

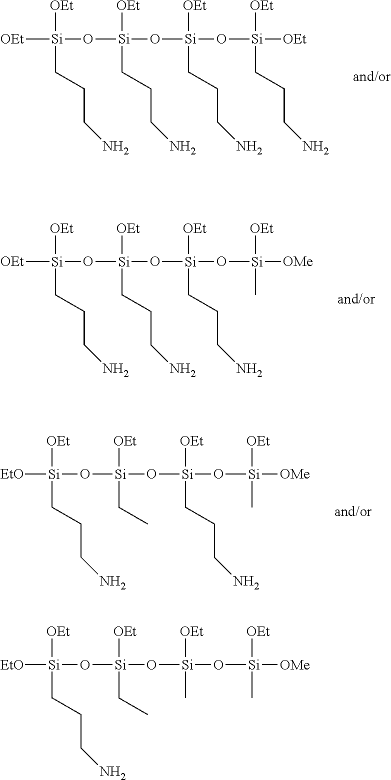 [SMILES: CCO[Si](CCCN)(OCC)O[Si](CCCN)(OCC)O[Si](CCCN)(OCC)O[Si](CCCN)(OCC)OCC ; CCO[Si](C)(OC)O[Si](CCCN)(OCC)O[Si](CCCN)(OCC)O[Si](CCCN)(OCC)OCC ; CCO[Si](C)(OC)O[Si](CCCN)(OCC)O[Si](CC)(OCC)O[Si](CCCN)(OCC)OCC ; CCO[Si](C)(OC)O[Si](C)(OCC)O[Si](CC)(OCC)O[Si](CCCN)(OCC)OCC]

In a further preferred embodiment, the silanes (c), which may be comprising at least one structural unit of formula (III) may also be ring-shaped oligomeric compounds.

The ring-shaped silane condensates include structural units of the formula (III), whereby the ring size determines the number of structural units of the formula (III). Each of the following silane condensates with 4-silane units comprises four structural units of formula (III).

Possible condensation reactions to the ring-shaped silane condensate with 4 silane units, shown based on the mixture (3-aminopropyl)triethoxysilane and methyltrimethoxy silane):

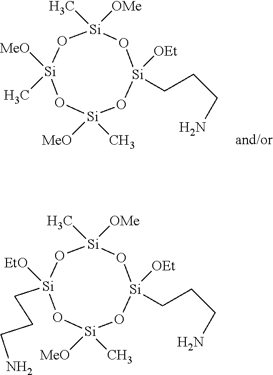 [SMILES: CCO[Si]1(CCCN)O[Si](C)(OC)O[Si](C)(OC)O[Si](C)(OC)O1 ; CCO[Si]1(CCCN)O[Si](C)(OC)O[Si](CCCN)(OCC)O[Si](C)(OC)O1]

The presence of very specific silanes (c) comprising at least one structural unit of formula (III) has been shown to be particularly advantageous in terms of achieving good application properties. When the compositions as contemplated herein were used as a coloring agent, particularly intensively colored keratin materials could be obtained if the composition included at least one silane (c) comprising at least one structural unit of the formula (IIIa),

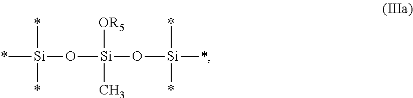

where
R5 represents a hydrogen atom or a C₁-C₆ alkyl group.

In the context of a further very particularly preferred embodiment, a composition as contemplated herein is wherein it comprises at least one silane (c) which comprises at least one structural unit of the formula (IIIa),

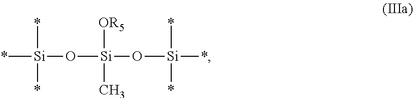

where
R5 represents a hydrogen atom or a C₁-C₆ alkyl group.

The radical R5 stands for a hydrogen atom or a C₁-C₆ alkyl group. Very preferably, R5 represents a hydrogen atom, a methyl group, or an ethyl group.

When the compositions as contemplated herein were used as a coloring agent, particularly intensively colored keratin materials could also be obtained if the composition included at least one silane (c) comprising at least one structural unit of the formula (IIIb),

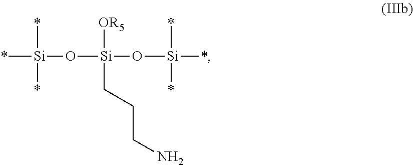

where
R5 represents a hydrogen atom or a C₁-C₆ alkyl group.

In the context of a further very particularly preferred embodiment, a composition as contemplated herein is wherein it comprises at least one silane (b) which comprises at least one structural unit of the formula (IIb),

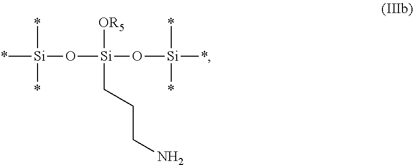

where
R5 represents a hydrogen atom or a C₁-C₆ alkyl group.

The radical R5 stands for a hydrogen atom or a C₁-C₆ alkyl group. Very preferably, R5 represents a hydrogen atom, a methyl group, or an ethyl group.

The best application properties were observed for compositions comprising both at least one silane (c) having at least one structural unit of formula (IIIa) and at least one silane (c) having at least one structural unit of formula (IIIb).

In an explicitly very particularly preferred embodiment, a composition as contemplated herein is wherein it comprises at least one silane (a) of the formula (IIIa) and at least one silane of the formula (IIIb)

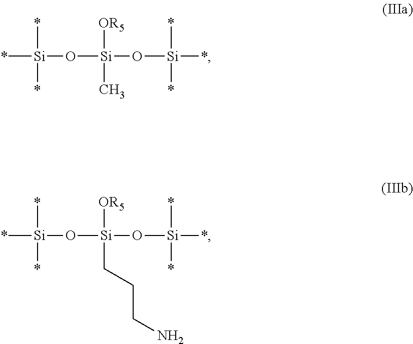

where
the radical R5 in the formula (IIIa) can be chosen independently of the radical R5 in the formula (IIIb) and independently represents a hydrogen atom or a C₁-C₆ alkyl group, particularly preferably a hydrogen atom, a methyl group, or an ethyl group.

With compositions as contemplated herein which—based on the total molar amount of all silicon compounds used in the composition—included one or more silanes (c) with a total molar proportion of about 22.0 to about 60.0 mol %, preferably of about 26.0 to about 56.0 mol %, further preferably of about 30.0 to about 42.0 mol % and very particularly preferably of about 34.0 to about 48.0 mol % of structural units of the formula (III), very particularly good and intensive color results were obtained.

Within the scope of a further embodiment, quite particularly preferred is a A composition comprising—based on the total molar amount of all silicon compounds used in the composition—one or more silanes (c) having a total molar content of from about 22.0 to about 60.0 mol %, preferably from about 26.0 to about 56.0 mol %, further preferably from about 30.0 to about 42.0 mol % and very particularly preferably from about 34.0 to about 48.0 mol % of structural units of the formula (III).

It is further particularly preferred if the composition as contemplated herein comprises the silanes having at least one structural unit of formula (IIIa) in certain molar proportions. With compositions as contemplated herein which—based on the total molar amount of all silicon compounds used in the composition—included one or more silanes (c) with a total molar proportion of about 16.0 to about 38.0 mol %, preferably of about 18.0 to about 36.0 mol %, further preferably of about 20.0 to about 34.0 mol % and very particularly preferably of about 22.0 to about 32.0 mol % of structural units of the formula (IIIa), very particularly good and intensive color results were obtained.

Within the scope of a further embodiment, quite particularly preferred is a A composition comprising—based on the total molar amount of all silicon compounds used in the composition—one or more silanes (c) having a total molar content of from about 16.0 to about 38.0 mol %, preferably from about 18.0 to about 36.0 mol %, further preferably from about 20.0 to about 34.0 mol % and very particularly preferably from about 22.0 to about 32.0 mol % of structural units of the formula (IIIa).

It is further particularly preferred if the composition as contemplated herein comprises the silanes having at least one structural unit of formula (IIIb) in certain molar proportions. With compositions as contemplated herein which—based on the total molar amount of all silicon compounds used in the composition—included one or more silanes (c) in a total molar proportion of about 6.0 to about 22.0 mol %, preferably of about 8.0 to about 20.0 mol %, further preferably of about 10.0 to about 18.0 mol % and very particularly preferably of about 12.0 to about 16.0 mol % of structural units of the formula (IIIb), very particularly good and intensive color results were obtained.

Within the scope of a further embodiment, quite particularly preferred is a A composition comprising—based on the total molar amount of all silicon compounds used in the composition—one or more silanes (c) in a total molar proportion of from about 6.0 to about 22.0 mol %, preferably from about 8.0 to about 20.0 mol %, further preferably from about 10.0 to about 18.0 mol % and very particularly preferably from about 12.0 to about 16.0 mol % of structural units of the formula (IIIb).

The molar amount of the silanes (c) with a structural unit of the formula (III) included in the composition as contemplated herein is determined, as described above, very preferably by employing quantitative 29-silicon NMR spectroscopy.

### Silanes (d) Comprising at Least One Structural Unit of Formula (IV)

A further typical feature of the compositions as contemplated herein is their content of at least one silane (d) which comprises at least one structural unit of the formula (IV),

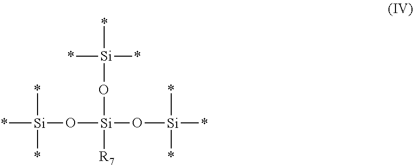

where
R7 is a C₁-C₈ alkyl group or an amino-C₁-C₈ alkyl group.

The silanes (d) have at least one structural unit of the formula (IV). The structural units of formula (IV) are triple cross-linked silanes which can be obtained, for example, by the complete cross-linking of the monomeric C₁-C₆ alkoxysilanes.

In other words, the structural units of formula (IV) are formed by condensing all three C1-C6 alkoxy groups of a silane of formula (I)—optionally after prior hydrolysis—with further silicon atoms, with elimination of water or alcohol, so that a branched, net-like structure is formed. The central silicon atom, which carries the radical R7, is bound in this way to three other silicon atoms via three oxygen atoms.

In the structural unit of formula (IV), the radical R7 represents a C₁-C₈ alkyl group or an amino-C₁-C₈ alkyl group. Very preferably, the radical R7 represents a methyl group, an ethyl group, an n-hexyl group, an n-octyl group and a 3-aminopropyl group.

The bonds in the structural unit of formula (IV), which start from the three terminal silicon atoms and are marked with an asterisk, represent the further free bond valences of these silicon atoms, i.e., these Si atoms each have three further bonds, which preferably go to a further carbon or to an oxygen atom.

The structural unit of formula (IV) is thus exemplified by the fact that it comprises a triple-crosslinked silicon atom which has three further bonds to three silicon atoms via three oxygen atoms

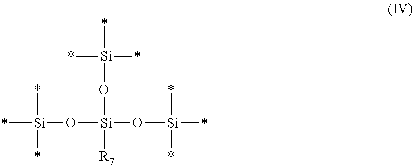

The silanes (d) with at least one structural unit of formula (IV) are oligomers with at least 4 Si atoms, i.e., the silanes (d) were obtained by condensation of at least four monomeric C₁-C₆ alkoxysilanes.

In a particularly preferred embodiment, the silanes (d), which may be which comprise at least one structural unit of the formula (IV) are, for example, crosslinked oligomeric compounds which can be formed via the subsequent reactions:

Possible condensation reactions starting from the mixture of (3-aminopropyl)triethoxysilane and methyltrimethoxysilane can lead, for example, to the following silanes (d):

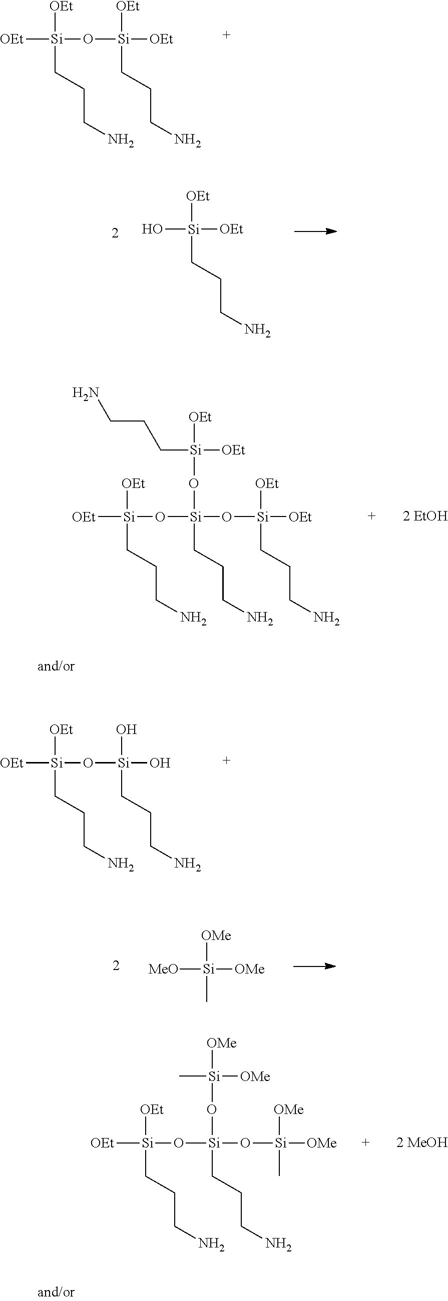

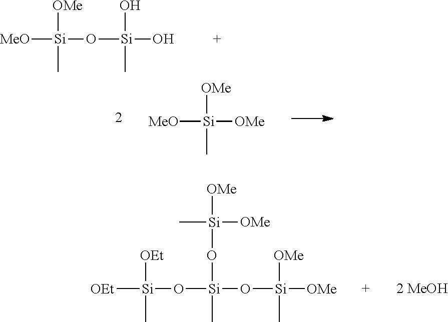

Possible condensation reactions starting from the mixture of (3-aminopropyl)triethoxysilane and methyltrimethoxysilane can lead, for example, to the following silanes (d):

Analogous to the reactions shown above, condensation to higher oligomers with more than 4 silane units is also possible.

The presence of very specific silanes (d) comprising at least one structural unit of formula (IV) has been shown to be particularly advantageous in terms of achieving good application properties. When the compositions as contemplated herein were used as a coloring agent, particularly intensively colored keratin materials could be obtained if the composition included at least one silane (d) comprising at least one structural unit of the formula (IVa),

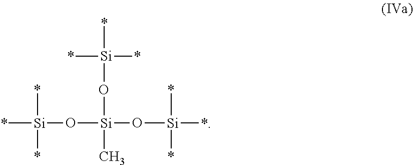

In the context of a further very particularly preferred embodiment, a composition as contemplated herein is wherein it comprises at least one silane (d) which comprises at least one structural unit of the formula (IVa),

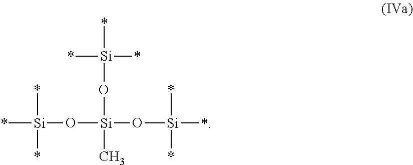

When the compositions as contemplated herein were used as a coloring agent, particularly intensively colored keratin materials could also be obtained if the composition included at least one silane (c) which comprises at least one structural unit of the formula (IVb),

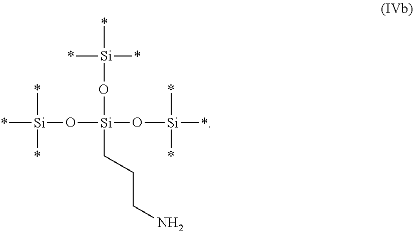

In the context of a further very particularly preferred embodiment, a composition as contemplated herein is wherein it comprises at least one silane (d) comprising at least one structural unit of the formula (IVb),

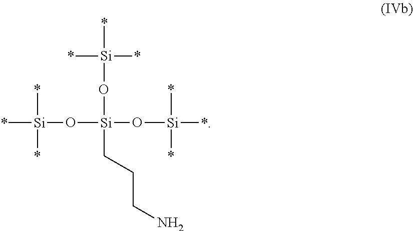

With compositions as contemplated herein which—based on the total molar amount of all silicon compounds used in the composition—included one or more silanes (d) with a total molar proportion of about 18.0 to about 34.0 mol %, preferably of about 20.0 to about 32.0 mol %, further preferably of about 22.0 to about 30.0 mol % and very particularly preferably of about 23.0 to about 28.0 mol % of structural units of the formula (IV), very particularly good and intensive color results were obtained.

Within the scope of a further embodiment, quite particularly preferred is a A composition comprising—based on the total molar amount of all silicon compounds used in the composition—one or more silanes (d) having a total molar content of from about 18.0 to about 34.0 mol %, preferably from about 20.0 to about 32.0 mol %, further preferably from about 22.0 to about 30.0 mol % and very particularly preferably from about 23.0 to about 28.0 mol % of structural units of the formula (IV).

The molar amount of the silanes (c) with a structural unit of the formula (III) included in the composition as contemplated herein is determined, as described above, very preferably by employing quantitative 29-silicon NMR spectroscopy.

### Preparation of Compositions Comprising the Silanes (a), (b), (c) and (d)

The compositions as contemplated herein contain a mixture of the monomeric or oligomeric silanes (a), (b), (c) and (d).

The preparation of these compositions is possible, for example, by reacting the monomeric C₁-C₆ alkoxy silanes (a) of the formula (I) with water, the selected amounts of C₁-C₆ alkoxy silanes and water being co-determinant for the proportions in which the silanes (a), (b), (c) and (d) are formed.

The reaction of the organic C₁-C₆ alkoxy silanes with water can take place in different ways. One possibility is to prepare the desired amount of water in the reaction vessel or reactor and then add the C₁-C₆ alkoxy silane(s) (a1) and (a2). In another embodiment, the appropriate amounts of C₁-C₆ alkoxy silanes of the formula (I) are first introduced into a reaction vessel or reactor, and the desired amount of water is then added.

The water can be added continuously, in partial quantities or directly as a total quantity. To ensure the required temperature control, the reaction mixture is preferably cooled and/or the amount and rate of water added is adjusted. To maintain the desired temperature ranges, it has been found to be particularly suitable to add the necessary amount of water continuously dropwise to a mixture of silanes of formulae (I). Depending on the amount of silanes used, the addition and reaction can take place over a period of about 2 minutes to about 72 hours.

Due to the high reactivity of the C₁-C₆ alkoxy silanes of formula (I), complex mixtures of hydrolyzed or condensed silanes are formed when they react with water. The exact composition of these mixtures is determined primarily by the molar amounts in which silanes (a) of formula (I) and water are used, respectively, in the reaction leading to the mixture of (a), (b), (c) and (d9.

As previously described, the work leading to the present disclosure has shown that, when the composition as contemplated herein was applied to the keratin material, a stable and resistant coating could be produced when the C₁-C₆ alkoxy silanes (a), (b), (c) and (d) were present in the composition in the molar ratios to each other described previously as preferred and most preferred.

In addition, the mixture of silanes (a) to (d) is very preferably present in certain ranges of amounts in the composition as contemplated herein. Particularly good results were obtained when the composition comprises—based on its total weight—one or more silanes (a) to (d) in a total amount of from about 30.0 to about 85.0% by weight, preferably from about 35.0 to about 80.0% by weight, more preferably from about 40.0 to about 75.0% by weight, still more preferably from about 45.0 to about 70.0% by weight, and most preferably from about 50.0 to about 65.0% by weight.

In the context of a further embodiment, a composition as contemplated herein comprising—based on the total weight of the composition—one or more silanes (a) to (d) in a total amount of from about 30.0 to about 85.0% by weight, preferably from about 35.0 to about 80.0% by weight, more preferably from about 40.0 to about 75.0% by weight, still more preferably from about 45.0 to about 70.0% by weight and very particularly preferably from about 50.0 to about 65.0% by weight is quite particularly preferred.

The preparation of the mixture of the organic C₁-C₆ alkoxy silanes of formula (I) and water can be carried out, for example, in a reaction vessel or a reactor, preferably a double-walled reactor, a reactor with external heat exchanger, a tubular reactor, a reactor with thin-film evaporator, a reactor with falling-film evaporator and/or a reactor with attached condenser.

A reaction vessel that is very suitable for smaller preparations is, for example, a glass flask commonly used for chemical reactions with a capacity of about 1 liter, about 3 liters or about 5 liters, such as a 3-liter single-neck or multi-neck flask with ground joints.

A reactor is a confined space (container, vessel) that has been specially designed and manufactured to allow certain reactions to take place and be controlled under defined conditions.

For larger approaches, it has proven advantageous to carry out the reaction in reactors made of metal. Typical reactors may include, for example, an about 10-liter, 20-liter, or 50-liter capacity. Larger reactors for the production area can also include fill volumes of about 100-liters, about 500-liters, or about 1000-liters.

Double-wall reactors have two reactor shells or reactor walls, with a tempering fluid circulating in the area between the two walls. This enables particularly good adjustment of the temperature to the required values.

The use of reactors, in particular double-walled reactors with an enlarged heat exchange surface, has also proven to be particularly suitable, whereby the heat exchange can take place either through internal installations or using an external heat exchanger.

Content of C₁-C₆ Alcohols in the Composition

As described previously, mixing the reactive C₁-C₆ alkoxy silanes of formula (I) with water initiates a hydrolysis reaction in which the C₁-C₆ alkoxy groups located directly on the silicon atom are hydrolyzed and the corresponding C₁-C₆ alcohols are released.

The partially or completely hydrolyzed silanes formed during hydrolysis are also reactive compounds that can undergo subsequent reactions in which these silanes of different degrees of hydrolysis condense with each other.

As can be seen from the reaction schemes shown previously, the condensation reactions in turn release either C₁-C₆ alcohols (for example ethanol and/or methanol) or water, with the amount of C₁-C₆ alcohols/water released depending on the extent to which the equilibrium of the above reactions is on the side of the condensates.

The extent of the condensation reaction, in turn, is partly determined by the amount of water initially added. Preferably, the amount of water is such that the condensation is a partial condensation, where “partial condensation” or “partial condensation” in this context means that not all the condensable groups of the silanes presented react with each other, so that the resulting organic silicon compound still has on average at least one hydrolysable/condensable group per molecule.

The amounts of C₁-C₆ alcohols and water released in the condensation reaction can be removed from the reaction mixture by various separation methods (for example, distillation).

When applying the composition as contemplated herein on the keratin material, the generation of a stable, coherent, and uniform coating is the basic prerequisite for achieving the desired application properties. Intense and long-lasting colorations can be obtained especially if the colorant compounds can be integrated into an appropriately resistant coating. It has been found that it is essential for this purpose to keep the content of C₁-C₆ alcohols in the composition as contemplated herein as low as possible.

For this reason, there is a requirement that the composition as contemplated herein comprises one or more C₁-C₆ alcohols in a total amount of about 0.001 to about 10.0% by weight.

For the purposes of the invention, C₁-C₆ alcohols are alcohols having one or more hydroxy groups comprising from 1 to 6 carbon atoms. These alcohols can be linear or branched, saturated or mono- or polyunsaturated. By C₁-C₆ mono-alcohols are meant the alcohols chosen from methanol, ethanol, n-propanol, isopropanol, 1-butanol, 2-butanol, 1-pentanol, 2-pentanol, 3-pentanol, 1-hexanol, 2-hexanol and 3-hexanol. C₁-C₆ alcohols with two hydroxyl groups include ethylene glycol, 1,2-propanediol and 1,3-propanediol. For example, a C₁-C₆ alcohol with three hydroxyl groups is glycerol.

With preparations whose total content of C₁-C₆ alcohols was about 10.0% by weight, dyeing's with sufficiently high color intensity could be obtained when applied to the keratin material.

However, even better results were obtained when the total content of C₁-C₆ alcohols—based on the total weight of the composition—could be limited to a total amount of from about 0.01 to about 9.0% by weight, preferably from about 0.1 to about 8.0% by weight, more preferably from about 0.5 to about 7.0% by weight and most preferably from about 0.5 to about 4.0% by weight.

In a further embodiment, very particularly preferred is a composition comprising, based on the total weight of the composition, one or more C₁-C₆ alcohols in a total amount of from about 0.01 to about 9.0% by weight, preferably from about 0.1 to about 8.0% by weight, more preferably from about 0.5 to about 7.0% by weight, and most preferably from about 0.5 to about 5.0% by weight.

The determination of the content of C₁-C₆ alcohols in the composition as contemplated herein can be carried out by employing various analytical methods. One possibility is measurement by GC-MS. Gas chromatography with mass spectrometry coupling is the coupling of a gas chromatograph (GC) with a mass spectrometer (MS). The overall procedure or instrument coupling is also referred to as GC-MS, GC/MS or GCMS for short

To determine the content of C₁-C₆ alcohols, a sample of the composition can be analyzed by gas chromatography, for example, in a double determination on a nonpolar column. Identification of the assigned components can be performed by mass spectrometry using library comparison spectra (e.g., NIST or Wiley). The mean value is formed from each of the double determinations. Quantification can be performed, for example, by employing internal standard calibration (e.g., with methyl isobutyl ketone).

As already described, C₁-C₆ alkoxysilanes of the formula (I) which carry methoxysilane or ethoxysilane groups are very preferably used in the process as contemplated herein. These have the advantage that methanol and ethanol are released during hydrolysis and condensation, respectively, which can be easily removed from the reaction mixture by vacuum distillation due to their boiling points.

In a further embodiment, very particularly preferred is a composition comprising, based on the total weight of the composition, from about 0.01 to about 9.0% by weight, preferably from about 0.1 to about 8.0% by weight, more preferably from about 0.5 to about 7.0% by weight, and very particularly preferably from about 0.5 to about 4.0% by weight of ethanol.

Compliance with the maximum amounts of C₁-C₆ alcohols described above can be achieved, for example, by removing the C₁-C₆ alcohols from the reaction mixture. A particularly preferred method of removing the C₁-C₆ alcohols by distillation.

### Water Content in the Preparation

As the previously shown reaction schemes indicate, too high a water content can also shift the reaction equilibrium from the side of the silane condensates back to the side of the monomeric silanes. Without being limited to this theory, it is assumed in this context that above all the presence of a sufficiently high amount of oligomeric silane condensates is essential for achieving a uniform and resistant coating on the keratin material, which again is the basic prerequisite for producing dyeing results with sufficiently high intensity.

For this reason, it is essential to the invention to limit the water content in the composition as contemplated herein to a value of about 0.001 to about 10.0% by weight of water. With preparations whose water content was 10.0 wt. %, colorations with sufficiently high color intensity could be obtained when applied to the keratin material.

However, even better results were obtained when the composition included—based on the total weight of the composition—about 0.01 to about 9.0 wt. %, preferably about 0.1 to about 7.0 wt. %, further preferably about 0.2 to about 5.0 wt. % and most preferably about 0.5 to about 3.0 wt. % water.

In a further embodiment, very particularly preferred is a composition comprising, based on the total weight of the composition, from about 0.01 to about 9.0% by weight, preferably from about 0.1 to about 7.0% by weight, more preferably from about 0.2 to about 5.0% by weight, and very particularly preferably from about 0.5 to about 3.0% by weight of water.

The determination of the water content in the composition as contemplated herein can be carried out by employing various known analytical methods. One possibility is measurement by GC-MS. Gas chromatography with mass spectrometry coupling is the coupling of a gas chromatograph (GC) with a mass spectrometer (MS). The overall procedure or instrument coupling is also abbreviated as GC-MS, GC/MS or GCMS. Another possibility is to determine the water content by titration, for example by Karl-Fischer titration.

Another possibility is the determination of the content of water via quantitative NMR spectra, via quantitative 1H-NMR spectra.

### Other Ingredients in the Composition

Optionally, the compositions as contemplated herein may also contain one or more further cosmetic ingredients.

The cosmetic ingredients optionally usable in the composition as contemplated herein may be any suitable ingredients to impart further beneficial properties to the composition. For example, a solvent, a thickening or film-forming polymer, a surface-active compound from the group of nonionic, cationic, anionic, or zwitterionic/amphoteric surfactants, the coloring compounds from the group of pigments, the direct dyes, oxidation dye precursors, fatty components from the group of C₈-C₃₀ fatty alcohols, hydrocarbon compounds, fatty acid esters, acids and bases belonging to the group of pH regulators, perfumes, preservatives, plant extracts and protein hydrolysates.

The selection of these other substances will be made by the specialist according to the desired properties of the agents. Regarding other optional components and the quantities of these components used, explicit reference is made to the relevant manuals known to the specialist.

In this context, it has proven to be particularly preferred to use a cosmetic ingredient in the composition as contemplated herein which further improves the stability, in particular the storage stability, of the keratin treatment agent. In this context, the addition of one or more cosmetic ingredients selected from the group of hexamethyldisiloxane, octamethyltrisiloxane, decamethyltetrasiloxane, hexamethylcyclotrisiloxane, octamethylcyclotetrasiloxane and/or decamethylcyclopentasiloxane has been shown to be particularly beneficial in terms of increasing the stability of the composition.

In another very particularly preferred embodiment, a composition as contemplated herein is wherein it comprises.

comprises one or more cosmetic ingredients selected from the group of hexamethyldisiloxane, octamethyltrisiloxane, decamethyltetrasiloxane, hexamethylcyclotrisiloxane, octamethylcyclotetrasiloxane and/or decamethylcyclopentasiloxane. Hexamethyldisiloxane has the CAS number 107-46-0 and can be purchased commercially from Sigma-Aldrich, for example.

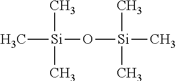

Octamethyltrisiloxane has the CAS number 107-51-7 and is also commercially available from Sigma-Aldrich.

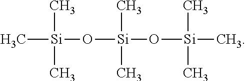

Decamethyltetrasiloxane carries the CAS number 141-62-8 and is also commercially available from Sigma-Aldrich.

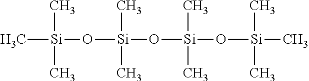

### Hexamethylcyclotrisiloxane has the CAS No. 541-05-9.

### Octamethylcyclotetrasiloxane has the CAS No. 556-67-2.

### Decamethylcyclopentasiloxane has the CAS No. 541-02-6.

To obtain compositions with particularly high storage stability, the addition of a cosmetic ingredient selected from the group of hexamethyldisiloxane, octamethyltrisiloxane, decamethyltetrasiloxane, hexamethylcyclotrisiloxane, octamethylcyclotetrasiloxane and decamethylcyclopentasiloxane has proved to be quite preferred.

In a further embodiment, very particularly preferred, a device as contemplated herein is a

composition comprising

one or more cosmetic ingredients selected from the group of hexamethyldisiloxane, octamethyltrisiloxane, decamethyltetrasiloxane, hexamethylcyclotrisiloxane, octamethylcyclotetrasiloxane and decamethylcyclopentasiloxane.

Explicitly, the use of hexamethyldisiloxane is particularly preferred since compositions with this ingredient have proven to be particularly stable. Preferably, the composition as contemplated herein comprises about 10.0 to about 50.0 wt. %, preferably about 15.0 to about 45.0 wt. %, more preferably about 20.0 to about 40.0 wt. %, still more preferably about 25.0 to about 35.0 wt. %, and most preferably about 31.0 to about 34.0 wt. % hexamethyldisiloxane.

In a further embodiment, very particularly preferred, a device as contemplated herein is a

Composition comprising—based on the total weight of the composition—about 10.0 to about 50.0 wt. %, preferably about 15.0 to about 45.0 wt. %, further preferably about 20.0 to about 40.0 wt. %, still further preferably about 25.0 to about 35.0 wt. % and most preferably about 31.0 to about 34.0 wt. % hexamethyldisiloxane.

### pH Values of the Preparations

In further experiments, it has been found that the pH of the composition as contemplated herein can also have an influence on the condensation reaction. It was found that alkaline pH values in particular stop condensation at the oligomer stage. The more acidic the reaction mixture, the faster the condensation appears to proceed and the higher the molecular weight of the silane condensates formed during condensation. For this reason, it is preferred if the composition has a pH of from about 7.0 to about 12.0, preferably from about 7.5 to about 11.5, more preferably from about 8.5 to about 11.0, and most preferably from about 9.0 to about 11.0.

The water content of the composition is at most about 10.0% by weight and is preferably set even lower. Particularly in the case of compositions with a very low water content, measuring the pH with the usual methods known from the prior art (pH value measurement by employing glass electrodes via combination electrodes or via pH indicator paper) can prove difficult. For this reason, the pH values as contemplated herein are those obtained after mixing or diluting the composition in a 1:1 ratio by weight with distilled water.

Accordingly, the corresponding pH is measured after, for example, about 50 g of the composition as contemplated herein has been mixed with about 50 g of distilled water.

In a further particularly preferred embodiment, a composition as contemplated herein is

composition wherein it has a pH of from about 7.0 to about 12.0, preferably from about 7.5 to about 11.5, more preferably from about 8.5 to about 11.0 and most preferably from about 9.0 to about 11.0, after dilution with distilled water in a weight ratio of about 1:1.

To adjust this alkaline pH, it may be necessary to add an alkalizing agent and/or acidifying agent to the reaction mixture. The pH values for the purposes of the present disclosure are pH values measured at a temperature of about 22° C.

For example, ammonia, alkanolamines and/or basic amino acids can be used as alkalizing agents.

Alkanolamines may be selected from primary amines having a C2-C6 alkyl parent bearing at least one hydroxyl group. Preferred alkanolamines are selected from the group formed by 2-aminoethan-1-ol (monoethanolamine), 3-aminopropan-1-ol, 4-aminobutan-1-ol, -aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-amino-2-methylpropan-2-ol, 3-aminopropan-1,2-diol, 2-amino-2-methylpropan-1,3-diol.

For the purposes of the invention, an amino acid is an organic compound comprising in its structure at least one protonatable amino group and at least one —COOH or one —SO₃H group. Preferred amino acids are aminocarboxylic acids, especially α-(alpha)-aminocarboxylic acids and ω-aminocarboxylic acids, whereby α-aminocarboxylic acids are particularly preferred.

As contemplated herein, basic amino acids are those amino acids which have an isoelectric point pI of greater than about 7.0.

Basic α-aminocarboxylic acids contain at least one asymmetric carbon atom. In the context of the present disclosure, both possible enantiomers can be used equally as specific compounds or their mixtures, especially as racemates. However, it is particularly advantageous to use the naturally preferred isomeric form, usually in L-configuration.

The basic amino acids are preferably selected from the group formed by arginine, lysine, ornithine and histidine, especially preferably arginine and lysine. In another particularly preferred embodiment, an agent as contemplated herein is therefore wherein the alkalizing agent is a basic amino acid from the group arginine, lysine, ornithine and/or histidine.

In addition, inorganic alkalizing agents can also be used. Inorganic alkalizing agents usable as contemplated herein are preferably selected from the group formed by sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Particularly preferred alkalizing agents are ammonia, 2-aminoethan-1-ol (monoethanolamine), 3-aminopropan-1-ol, 4-aminobutan-1-ol, 5-aminopentan-1-ol, 1-aminopropan-2-ol, 1-aminobutan-2-ol, 1-aminopentan-2-ol, 1-aminopentan-3-ol, 1-aminopentan-4-ol, 3-amino-2-methylpropan-1-ol, 1-Amino-2-methylpropan-2-ol, 3-aminopropan-1,2-diol, 2-amino-2-methylpropan-1,3-diol, arginine, lysine, ornithine, histidine, sodium hydroxide, potassium hydroxide, calcium hydroxide, barium hydroxide, sodium phosphate, potassium phosphate, sodium silicate, sodium metasilicate, potassium silicate, sodium carbonate and potassium carbonate.

Besides the alkalizing agents described above, experts are familiar with common acidifying agents for fine adjustment of the pH-value. As contemplated herein, preferred acidifiers are pleasure acids, such as citric acid, acetic acid, malic acid or tartaric acid, as well as diluted mineral acids.

### Multi-Component Packaging Unit (Kit-of-Parts)

The composition described above is the storage-stable form of the silane blend (i.e., the silane blend), which preferably has a particularly low water content.

For use in a process for the treatment of keratinous material, for the treatment of keratinous fibers, the user must convert this storage-stable blend into an agent ready for use. The ready-to-use agent usually has a higher water content.

For this purpose, the user may mix the previously described low-water composition (i.e., the blend of silane condensates) with one or more other compositions shortly before use. To increase user convenience, all required compositions can be provided to the user in the form of a multi-component packaging unit (kit-of-parts).

Explicitly, the compositions show particularly good suitability when used in a dyeing process.

When applying the compositions of the invention to a dyeing process, one or more colorant compounds may be used. The color-imparting compound(s) may, for example, be included in a separately prepared cosmetic composition (B).

A second object of the present disclosure is a multi-component packaging unit (kit-of-parts) for dyeing keratinous material, in particular human hair, which separately includes

a first packaging unit comprising a cosmetic composition (A) and

a second packaging unit comprising a cosmetic composition (B),

where

the cosmetic composition (A) is a composition as disclosed in detail in the description of the first subject matter of the invention, and

the cosmetic composition (B) comprises at least one colorant compound selected from the group of pigments and/or direct dyes.

The coloring compound or compounds can preferably be selected from pigments, substantive dyes, oxidation dyes, photochromic dyes and thermochromic dyes, particularly preferably from pigments and/or substantive dyes.

Pigments within the meaning of the present disclosure are coloring compounds which have a solubility in water at about 25° C. of less than about 0.5 g/L, preferably less than about 0.1 g/L, even more preferably less than about 0.05 g/L. Water solubility can be determined, for example, by the method described below: about 0.5 g of the pigment are weighed in a beaker. A stir-fish is added. Then one liter of distilled water is added. This mixture is heated to about 25° C. for one hour while stirring on a magnetic stirrer. If undissolved components of the pigment are still visible in the mixture after this period, the solubility of the pigment is below about 0.5 g/L. If the pigment-water mixture cannot be assessed visually due to the high intensity of the possibly finely dispersed pigment, the mixture is filtered. If a proportion of undissolved pigments remains on the filter paper, the solubility of the pigment is below about 0.5 g/L.

Suitable color pigments can be of inorganic and/or organic origin.

In a preferred embodiment, an agent as contemplated herein is wherein it comprises (b) at least one coloring compound from the group of inorganic and/or organic pigments.

Preferred color pigments are selected from synthetic or natural inorganic pigments. Inorganic color pigments of natural origin can be produced, for example, from chalk, ochre, umber, green earth, burnt Terra di Siena or graphite. Furthermore, black pigments such as iron oxide black, colored pigments such as ultramarine or iron oxide red as well as fluorescent or phosphorescent pigments can be used as inorganic color pigments.

Particularly suitable are colored metal oxides, hydroxides and oxide hydrates, mixed-phase pigments, sulfur-comprising silicates, silicates, metal sulfides, complex metal cyanides, metal sulphates, chromates and/or molybdates. Preferred color pigments are black iron oxide (CI 77499), yellow iron oxide (CI 77492), red and brown iron oxide (CI 77491), manganese violet (CI 77742), ultramarine (sodium aluminum sulfo silicates, CI 77007, pigment blue 29), chromium oxide hydrate (CI77289), iron blue (ferric ferrocyanides, CI77510) and/or carmine (cochineal).

Colored pearlescent pigments are also particularly preferred colorants from the group of pigments as contemplated herein. These are usually mica- and/or mica-based and can be coated with one or more metal oxides. Mica belongs to the layer silicates. The most important representatives of these silicates are muscovite, phlogopite, paragonite, biotite, lepidolite and margarite. To produce the pearlescent pigments in combination with metal oxides, the mica, mainly muscovite or phlogopite, is coated with a metal oxide.

As an alternative to natural mica, synthetic mica coated with one or more metal oxides can also be used as pearlescent pigment. Especially preferred pearlescent pigments are based on natural or synthetic mica (mica) and are coated with one or more of the metal oxides mentioned above. The color of the respective pigments can be varied by varying the layer thickness of the metal oxide(s).

In a further preferred embodiment, an agent as contemplated herein is wherein it comprises (b) at least one colorant compound from the group of pigments selected from the group of colored metal oxides, metal hydroxides, metal oxide hydrates, silicates, metal sulfides, complex metal cyanides, metal sulfates, bronze pigments and/or from mica- or mica-based colorant compounds coated with at least one metal oxide and/or a metal oxychloride.

In a further preferred embodiment, a composition as contemplated herein is wherein it comprises (b) at least one colorant compound selected from mica- or mica-based pigments reacted with one or more metal oxides selected from the group of titanium dioxide (CI 77891), black iron oxide (CI 77499), yellow iron oxide (CI 77492), red and/or brown iron oxide (CI 77491, CI 77499), manganese violet (CI 77742), ultramarines (sodium aluminum sulfosilicates, CI 77007, Pigment Blue 29), chromium oxide hydrate (CI 77289), chromium oxide (CI 77288) and/or iron blue (ferric ferrocyanide, CI 77510).

Examples of particularly suitable color pigments are commercially available under the trade names Rona®, Colorona®, Xirona®, Dichrona® and Timiron® from Merck, Ariabel® and Unipure® from Sensient, Prestige® from Eckart Cosmetic Colors and Sunshine® from Sunstar.

Particularly preferred color pigments with the trade name Colorona® are, for example:

### Colorona Copper, Merck, MICA, CI 77491 (IRON OXIDES)

### Colorona Passion Orange, Merck, Mica, CI 77491 (Iron Oxides), Alumina

### Colorona Patina Silver, Merck, MICA, CI 77499 (IRON OXIDES), CI 77891 (TITANIUM DIOXIDE)

### Colorona RY, Merck, CI 77891 (TITANIUM DIOXIDE), MICA, CI 75470 (CARMINE)

### Colorona Oriental Beige, Merck, MICA, CI 77891 (TITANIUM DIOXIDE), CI 77491 (IRON OXIDES)

### Colorona Dark Blue, Merck, MICA, TITANIUM DIOXIDE, FERRIC FERROCYANIDE

### Colorona Chameleon, Merck, CI 77491 (IRON OXIDES), MICA

### Colorona Aborigine Amber, Merck, MICA, CI 77499 (IRON OXIDES), CI 77891 (TITANIUM DIOXIDE)

### Colorona Blackstar Blue, Merck, CI 77499 (IRON OXIDES), MICA

### Colorona Patagonian Purple, Merck, MICA, CI 77491 (IRON OXIDES), CI 77891 (TITANIUM DIOXIDE), CI 77510 (FERRIC FERROCYANIDE)

### Colorona Red Brown, Merck, MICA, CI 77491 (IRON OXIDES), CI 77891 (TITANIUM DIOXIDE)

### Colorona Russet, Merck, CI 77491 (TITANIUM DIOXIDE), MICA, CI 77891 (IRON OXIDES)

### Colorona Imperial Red, Merck, MICA, TITANIUM DIOXIDE (CI 77891), D&C RED NO. 30 (CI 73360)

### Colorona Majestic Green, Merck, CI 77891 (TITANIUM DIOXIDE), MICA, CI 77288 (CHROMIUM OXIDE GREENS)

### Colorona Light Blue, Merck, MICA, TITANIUM DIOXIDE (CI 77891), FERRIC FERROCYANIDE (CI 77510)

### Colorona Red Gold, Merck, MICA, CI 77891 (TITANIUM DIOXIDE), CI 77491 (IRON OXIDES)

### Colorona Gold Plus MP 25, Merck, MICA, TITANIUM DIOXIDE (CI 77891), IRON OXIDES (CI 77491)

### Colorona Carmine Red, Merck, MICA, TITANIUM DIOXIDE, CARMINE

### Colorona Blackstar Green, Merck, MICA, CI 77499 (IRON OXIDES)

### Colorona Bordeaux, Merck, MICA, CI 77491 (IRON OXIDES)

### Colorona Bronze, Merck, MICA, CI 77491 (IRON OXIDES)

### Colorona Bronze Fine, Merck, MICA, CI 77491 (IRON OXIDES)

### Colorona Fine Gold MP 20, Merck, MICA, CI 77891 (TITANIUM DIOXIDE), CI 77491 (IRON OXIDES)

### Colorona Sienna Fine, Merck, CI 77491 (IRON OXIDES), MICA

### Colorona Sienna, Merck, MICA, CI 77491 (IRON OXIDES)

Colorona Precious Gold, Merck, Mica, CI 77891 (Titanium dioxide), Silica, CI 77491 (Iron oxides), Tin oxide

### Colorona Sun Gold Sparkle MP 29, Merck, MICA, TITANIUM DIOXIDE, IRON OXIDES, MICA, CI 77891, CI 77491 (EU)

Colorona Mica Black, Merck, CI 77499 (Iron oxides), Mica, CI 77891 (Titanium dioxide)
Colorona Bright Gold, Merck, Mica, CI 77891 (Titanium dioxide), CI 77491 (Iron oxides)

### Colorona Blackstar Gold, Merck, MICA, CI 77499 (IRON OXIDES)

Other particularly preferred color pigments with the trade name Xirona® are for example:

### Xirona Golden Sky, Merck, Silica, CI 77891 (Titanium Dioxide), Tin Oxide

### Xirona Caribbean Blue, Merck, Mica, CI 77891 (Titanium Dioxide), Silica, Tin Oxide

### Xirona Kiwi Rose, Merck, Silica, CI 77891 (Titanium Dioxide), Tin Oxide

### Xirona Magic Mauve, Merck, Silica, CI 77891 (Titanium Dioxide), Tin Oxide.

In addition, particularly preferred color pigments with the trade name Unipure® are for example:

### Unipure Red LC 381 EM, Sensient CI 77491 (Iron Oxides), Silica

### Unipure Black LC 989 EM, Sensient, CI 77499 (Iron Oxides), Silica

### Unipure Yellow LC 182 EM, Sensient, CI 77492 (Iron Oxides), Silica

In a further embodiment, the features as contemplated herein may also contain (b) one or more coloring compounds from the group of organic pigments

The organic pigments as contemplated herein are correspondingly insoluble, organic dyes or color lacquers, which may be selected, for example, from the group of nitroso, nitro-azo, xanthene, anthraquinone, isoindolinone, isoindolinone, quinacridone, perinone, perylene, diketo-pyrrolopyorrole, indigo, thioindido, dioxazine and/or triarylmethane compounds.

Examples of particularly suitable organic pigments are carmine, quinacridone, phthalocyanine, sorghum, blue pigments with the Color Index numbers Cl 42090, CI 69800, CI 69825, CI 73000, CI 74100, CI 74160, yellow pigments with the Color Index numbers CI 11680, CI 11710, CI 15985, CI 19140, CI 20040, CI 21100, CI 21108, CI 47000, CI 47005, green pigments with the Color Index numbers CI 61565, CI 61570, CI 74260, orange pigments with the Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

In another particularly preferred embodiment, a composition as contemplated herein is wherein it comprises at least one colorant compound from the group of organic pigments selected from the group of carmine, quinacridone, phthalocyanine, sorghum, blue pigments having the Color Index numbers Cl 42090, CI 69800, CI 69825, CI 73000, CI 74100, CI 74160, yellow pigments having the Color Index numbers CI 11680, CI 11710, CI 15985, CI 19140, CI 20040, CI 21100, CI 21108, CI 47000, CI 47005, green pigments with Color Index numbers CI 61565, CI 61570, CI 74260, orange pigments with Color Index numbers CI 11725, CI 15510, CI 45370, CI 71105, red pigments with the Color Index numbers CI 12085, CI 12120, CI 12370, CI 12420, CI 12490, CI 14700, CI 15525, CI 15580, CI 15620, CI 15630, CI 15800, CI 15850, CI 15865, CI 15880, CI 17200, CI 26100, CI 45380, CI 45410, CI 58000, CI 73360, CI 73915 and/or CI 75470.

The organic pigment can also be a color paint. As contemplated herein, the term color lacquer means particles comprising a layer of absorbed dyes, the unit of particle and dye being insoluble under the above-mentioned conditions. The particles can, for example, be inorganic substrates, which can be aluminum, silica, calcium borosilate, calcium aluminum borosilicate or even aluminum.

For example, alizarin color varnish can be used.

Due to their excellent resistance to light and temperature, the use of the pigments in the features as contemplated herein is particularly preferred. It is also preferred if the pigments used have a certain particle size. This particle size leads on the one hand to an even distribution of the pigments in the formed polymer film and on the other hand avoids a rough hair or skin feeling after application of the cosmetic product. As contemplated herein, it is therefore advantageous if the at least one pigment has an average particle size D₅₀ of about 1.0 to about 50 μm, preferably about 5.0 to about 45 μm, preferably about 10 to about 40 μm, about 14 to about 30 μm. The mean particle size D₅₀, for example, can be determined using dynamic light scattering (DLS).

The pigment or pigments may be used in an amount of from about 0.001 to about 20% by weight, from about 0.05 to about 5% by weight, in each case based on the total weight of the composition as contemplated herein.

As colorant compounds, the compositions as contemplated herein may also contain one or more direct dyes. Direct-acting dyes are dyes that draw directly onto the hair and do not require an oxidative process to form the color. Direct dyes are usually nitrophenylene diamines, nitroaminophenols, azo dyes, anthraquinones, triarylmethane dyes or indophenols.

The direct dyes within the meaning of the present disclosure have a solubility in water (760 mmHg) at about 25° C. of more than about 0.5 g/L and are therefore not to be regarded as pigments. Preferably, the direct dyes within the meaning of the present disclosure have a solubility in water (760 mmHg) at about 25° C. of more than about 1.0 g/L. In particular, the direct dyes within the meaning of the present disclosure have a solubility in water (760 mmHg) at about 25° C. of more than about 1.5 g/L.

Direct dyes can be divided into anionic, cationic and nonionic direct dyes.

In a further preferred embodiment, an agent as contemplated herein is wherein it comprises as coloring compound (b) at least one anionic, cationic and/or non-ionic direct dye.

In a further preferred embodiment, an agent as contemplated herein is wherein it comprises (b) at least one anionic, cationic and/or non-ionic direct dye.

Suitable cationic direct dyes include Basic Blue 7, Basic Blue 26, Basic Violet 2 and Basic Violet 14, Basic Yellow 57, Basic Red 76, Basic Blue 16, Basic Blue 347 (Cationic Blue 347/Dystar), HC Blue No. 16, Basic Blue 99, Basic Brown 16, Basic Brown 17, Basic Yellow 57, Basic Yellow 87, Basic Orange 31, Basic Red 51 Basic Red 76

As non-ionic direct dyes, non-ionic nitro and quinone dyes and neutral azo dyes can be used. Suitable non-ionic direct dyestuffs are those listed under the international designations or Trade names HC Yellow 2, HC Yellow 4, HC Yellow 5, HC Yellow 6, HC Yellow 12, HC Orange 1, Disperse Orange 3, HC Red 1, HC Red 3, HC Red 10, HC Red 11, HC Red 13, HC Red BN, HC Blue 2, HC Blue 11, HC Blue 12, Disperse Blue 3, HC Violet 1, Disperse Violet 1, Disperse Violet 4, Disperse Black 9 known compounds, as well as 1,4-diamino-2-nitrobenzene, 2-amino-4-nitrophenol, 1,4-bis-(2-hydroxyethyl)-amino-2-nitrobenzene, 3-nitro-4-(2-hydroxyethyl)-aminophenol 2-(2-hydroxyethyl)amino-4,6-dinitrophenol, 4-[(2-hydroxyethyl)amino]-3-nitro-1-methylbenzene, 1-amino-4-(2-hydroxyethyl)-amino-5-chloro-2-nitrobenzene, 4-amino-3-nitrophenol, 1-(2′-ureidoethyl)amino-4-nitrobenzene, 2-[(4-amino-2-nitrophenyl)amino]benzoic acid, 6-nitro-1,2,3,4-tetrahydroquinoxaline, 2-hydroxy-1,4-naphthoquinone, picramic acid and its salts, 2-amino-6-chloro-4-nitrophenol, 4-ethylamino-3-nitrobenzoic acid and 2-chloro-6-ethylamino-4-nitrophenol.

Anionic direct dyes are also called acid dyes. Acid dyes are direct dyes that have at least one carboxylic acid group (—COOH) and/or one sulphonic acid group (—SO₃H). Depending on the pH value, the protonated forms (—COOH, —SO₃H) of the carboxylic acid or sulphonic acid groups are in equilibrium with their deprotonated forms (—OO⁻, —SO₃— present). The proportion of protonated forms increases with decreasing pH. If direct dyes are used in the form of their salts, the carboxylic acid groups or sulphonic acid groups are present in deprotonated form and are neutralized with corresponding stoichiometric equivalents of cations to maintain electro neutrality. Inventive acid dyes can also be used in the form of their sodium salts and/or their potassium salts.

The acid dyes within the meaning of the present disclosure have a solubility in water (760 mmHg) at about 25° C. of more than about 0.5 g/L and are therefore not to be regarded as pigments. Preferably the acid dyes within the meaning of the present disclosure have a solubility in water (760 mmHg) at about 25° C. of more than about 1.0 g/L.

The alkaline earth salts (such as calcium salts and magnesium salts) or aluminum salts of acid dyes often have a lower solubility than the corresponding alkali salts. If the solubility of these salts is below about 0.5 g/L (25° C., 760 mmHg), they do not fall under the definition of a direct dye.

An essential typical of acid dyes is their ability to form anionic charges, whereby the carboxylic acid or sulphonic acid groups responsible for this are usually linked to different chromophoric systems. Suitable chromophoric systems can be found, for example, in the structures of nitrophenylenediamines, nitroaminophenols, azo dyes, anthraquinone dyes, triarylmethane dyes, xanthene dyes, rhodamine dyes, oxazine dyes and/or indophenol dyes.

For example, one or more compounds from the following group can be selected as particularly well suited acid dyes: Acid Yellow 1 (D&C Yellow 7, Citronin A, Ext. D&C Yellow No. 7, Japan Yellow 403, CI 10316, COLIPA no B001), Acid Yellow 3 (COLIPA no: C 54, D&C Yellow No 10, Quinoline Yellow, E104, Food Yellow 13), Acid Yellow 9 (CI 13015), Acid Yellow 17 (CI 18965), Acid Yellow 23 (COLIPA no C 29, Covacap Jaune W 1100 (LCW), Sicovit Tartrazine 85 E 102 (BASF), Tartrazine, Food Yellow 4, Japan Yellow 4, FD&C Yellow No. 5), Acid Yellow 36 (CI 13065), Acid Yellow 121 (CI 18690), Acid Orange 6 (CI 14270), Acid Orange 7 (2-Naphthol orange, Orange II, CI 15510, D&C Orange 4, COLIPA no C015), Acid Orange 10 (C.I. 16230; Orange G sodium salt), Acid Orange 11 (CI 45370), Acid Orange 15 (CI 50120), Acid Orange 20 (CI 14600), Acid Orange 24 (BROWN 1; CI 20170; KATSU 201; no sodium salt; Brown No. 201; RESORCIN BROWN; ACID ORANGE 24; Japan Brown 201; D & C Brown No. 1), Acid Red 14 (C.I. 14720), Acid Red 18 (E124, Red 18; CI 16255), Acid Red 27 (E 123, CI 16185, C-Rot 46, Real red D, FD&C Red No. 2, Food Red 9, Naphthol red S), Acid Red 33 (Red 33, Fuchsia Red, D&C Red 33, CI 17200), Acid Red 35 (CI C.I. 18065), Acid Red 51 (CI 45430, Pyrosin B, Tetraiodfluorescein, Eosin J, Iodeosin), Acid Red 52 (CI 45100, Food Red 106, Solar Rhodamine B, Acid Rhodamine B, Red no 106 Pontacyl Brilliant Pink), Acid Red 73 (CI 27290), Acid Red 87 (Eosin, CI 45380), Acid Red 92 (COLIPA no C53, CI 45410), Acid Red 95 (CI 45425, Erythtosine, Simacid Erythrosine Y), Acid Red 184 (CI 15685), Acid Red 195, Acid Violet 43 (Jarocol Violet 43, Ext. D&C Violet no 2, C.I. 60730, COLIPA no C063), Acid Violet 49 (CI 42640), Acid Violet 50 (CI 50325), Acid Blue 1 (Patent Blue, CI 42045), Acid Blue 3 (Patent Blue V, CI 42051), Acid Blue 7 (CI 42080), Acid Blue 104 (CI 42735), Acid Blue 9 (E 133, Patent blue AE, Amido blue AE, Erioglaucin A, CI 42090, C.I. Food Blue 2), Acid Blue 62 (CI 62045), Acid Blue 74 (E 132, CI 73015), Acid Blue 80 (CI 61585), Acid Green 3 (CI 42085, Foodgreen 1), Acid Green 5 (CI 42095), Acid Green 9 (C.I. 42100), Acid Green 22 (C.I. 42170), Acid Green 25 (CI 61570, Japan Green 201, D&C Green No. 5), Acid Green 50 (Brilliant Acid Green BS, C.I. 44090, Acid Brilliant Green BS, E 142), Acid Black 1 (Black no 401, Naphthalene Black 10B, Amido Black 10B, CI 20 470, COLIPA no B15), Acid Black 52 (CI 15711), Food Yellow 8 (CI 14270), Food Blue 5, D&C Yellow 8, D&C Green 5, D&C Orange 10, D&C Orange 11, D&C Red 21, D&C Red 27, D&C Red 33, D&C Violet 2 and/or D&C Brown 1.

For example, the water solubility of anionic direct dyes can be determined in the following way. About 0.1 g of the anionic direct dye is placed in a beaker. A stir-fish is added. Then add about 100 ml of water. This mixture is heated to about 25° C. on a magnetic stirrer while stirring. It is stirred for about 60 minutes. The aqueous mixture is then visually assessed. If there are still undissolved residues, the amount of water is increased—for example in steps of 10 ml. Water is added until the amount of dye used is completely dissolved. If the dye-water mixture cannot be assessed visually due to the high intensity of the dye, the mixture is filtered. If a proportion of undissolved dyes remains on the filter paper, the solubility test is repeated with a higher quantity of water. If about 0.1 g of the anionic direct dye dissolves in about 100 ml water at about 25° C., the solubility of the dye is about 1.0 g/L.

Acid Yellow 1 is called 8-hydroxy-5,7-dinitro-2-naphthalenesulfonic acid disodium salt and has a solubility in water of at least about 40 g/L (25° C.).

Acid Yellow 3 is a mixture of the sodium salts of mono- and sisulfonic acids of 2-(2-quinolyl)-1H-indene-1,3(2H)-dione and has a water solubility of about 20 g/L (25° C.).
Acid Yellow 9 is the disodium salt of 8-hydroxy-5,7-dinitro-2-naphthalenesulfonic acid, its solubility in water is above about 40 g/L (25° C.).
Acid Yellow 23 is the trisodium salt of 4,5-dihydro-5-oxo-1-(4-sulfophenyl)-4-((4-sulfophenyl)azo)-1H-pyrazole-3-carboxylic acid and is highly soluble in water at 25° C.
Acid Orange 7 is the sodium salt of 4-[(2-hydroxy-1-naphthyl)azo]benzene sulphonate. Its water solubility is more than about 7 g/L (25° C.).
Acid Red 18 is the trinatirum salt of 7-hydroxy-8-[(E)-(4-sulfonato-1-naphthyl)-diazenyl]-1,3-naphthalene disulfonate and has a very high water solubility of more than 20% by weight.
Acid Red 33 is the diantrium salt of 5-amino-4-hydroxy-3-(phenylazo)-naphthalene-2,7-disulphonate, its solubility in water is about 2.5 g/L (25° C.).
Acid Red 92 is the disodium salt of 3,4,5,6-tetrachloro-2-(1,4,5,8-tetrabromo-6-hydroxy-3-oxoxanthen-9-yl)benzoic acid, whose solubility in water is indicated as greater than about 10 g/L (25° C.).
Acid Blue 9 is the disodium salt of 2-({4-[N-ethyl(3-sulfonatobenzyl]amino]phenyl}{4-[N-ethyl(3-sulfonatobenzyl)imino]-2,5-cyclohexadien-1-ylidene}methyl)-benzenesulfonate and has a solubility in water of more than about 20% by weight (25° C.).

Thermochromic dyes can also be used. Thermochromism involves the property of a material to change its color reversibly or irreversibly as a function of temperature. This can be done by changing both the intensity and/or the wavelength maximum.

Finally, it is also possible to use photochromic dyes. Photochromism involves the property of a material to change its color depending reversibly or irreversibly on irradiation with light, especially UV light. This can be done by changing both the intensity and/or the wavelength maximum.

The cosmetic composition (B) may contain—based on the total weight of the cosmetic composition (B)—one or more pigments in a total amount of from about 0.01 to about 10.0% by weight, preferably from about 0.1 to about 8.0% by weight, more preferably from about 0.2 to about 6.0% by weight, and most preferably from about 0.5 to about 4.5% by weight.

The cosmetic composition (B) may contain—based on the total weight of the cosmetic composition (B)—one or more direct dyes in a total amount of from about 0.01 to about 10.0% by weight, preferably from about 0.1 to about 8.0% by weight, more preferably from about 0.2 to about 6.0% by weight and most preferably from about 0.5 to about 4.5% by weight.

In addition to the preparations (A) and (B), the multicomponent packaging unit (kit-of-parts) as contemplated herein may also contain one or more further separately prepared preparations, for example a cosmetic composition (C) comprising at least one thickening polymer and/or a cosmetic composition (D) comprising at least one film-forming polymer.

In the context of a further embodiment, very particularly preferred is a multi-component packaging unit (kit-of-parts) comprising
a third packaging unit comprising a cosmetic composition (C), the cosmetic composition (C) comprising at least one thickening polymer

As a thickening polymer can be used, for example:

Vinylpyrrolidone/vinyl ester copolymers, such as those sold under the trademark Luviskol® (BASF). Luviskol® VA 64 and Luviskol® VA 73, each vinylpyrrolidone/vinyl acetate copolymers, are also preferred nonionic polymers.

Cellulose ethers, such as hydroxypropyl cellulose, hydroxyethyl cellulose and methyl hydroxypropyl cellulose, such as those sold under the trademarks Culminal® and Benecel® (AQUALON) and Natrosol® grades (Hercules).

Starch and its derivatives, especially starch ethers, for example Structure® XL (National Starch), a multifunctional, salt-tolerant starch;

Polyvinylpyrrolidones, such as those sold under the name Luviskol® (BASF).

In the context of a further embodiment, very particularly preferred is a multi-component packaging unit (kit-of-parts) comprising

a fourth packaging unit comprising a cosmetic composition (D), the cosmetic composition (D) comprising at least one film-forming polymer

As a film-forming polymer, at least one anionic polymer selected from the group of copolymers of acrylic acid, copolymers of methacrylic acid, homopolymers or copolymers of acrylic acid esters, homopolymers or copolymers of methacrylic acid esters, homopolymers or copolymers of acrylic acid amides of homopolymers or copolymers of methacrylic acid amides, of copolymers of vinylpyrrolidone, of copolymers of vinyl alcohol, of copolymers of vinyl acetate, of homopolymers or copolymers of ethylene, of homopolymers or copolymers of propylene, of homopolymers or copolymers of styrene, of polyurethanes, of polyesters and/or of polyamides.

Concerning the further preferred embodiments of the multicomponent packaging unit as contemplated herein, mutatis mutandis what has been said about the composition as contemplated herein applies.

### EXAMPLES

### 1. Preparation of the Silane Blends (Composition (A))

A reactor with a heatable/coolable outer shell and with a capacity of 10 liters was filled with 4.67 kg of methyltrimethoxysilane (34.283 mol). With stirring, 1.33 kg of (3-aminopropyl)triethoxysilane (6.008 mol) was then added. This mixture was stirred at 30° C. Subsequently, 670 ml of distilled water (37.18 mol) was added dropwise with vigorous stirring while maintaining the temperature of the reaction mixture at 30° C. under external cooling. After completion of the water addition, stirring was continued for another 10 minutes.

3-(Triethoxysilyl)propylamine molar mass=221.37 g/mol
Methyltrimethoxysilane molar mass=136.22 g/mol

total molar amount of silicon compounds used=34.283 mol+6.008 mol=40.291 mol

A vacuum of 180 mbar was then applied, and the reaction mixture was heated to a temperature of 65° C. Once the reaction mixture reached the temperature of 65° C., the reaction mixture was distilled over a period of 190 minutes. All substances distilled off were collected in a cooled receiver. The reaction mixture was then allowed to cool to room temperature.

In each case, 100 ml of the silane blend was filled into a bottle with a capacity of 100 ml and screw cap closure with seal. After filling, the bottles were tightly closed. The water content was less than 2.0% by weight.

Immediately after production, a sample was taken and examined by NMR spectroscopy (day 0).

Then the bottles were stored at 50° C. for 14 days. After a storage period of 7 days and after 14 days, samples were taken again and examined by NMR spectroscopy.

### 2. 29Si NMR Spectroscopy

### Solvent: Chloroform

### Device: Agilent, 600 MHz

Standard: TMS (tetramethylsilane)
Relaxation accelerator: Chromium(III) acetylacetonate
By using the relaxation accelerator, the intregrals of the individual signals became comparable with each other. The sum over all integrals was set equal to 100 mol %.
For the quantitative determination, the integrated area of each individual signal was related to the total sum over all integrals.

In the range of the monomeric compounds (compounds of formula (I)), the singly cross-linked compounds (silanes with structural unit of formula (II)) and the doubly cross-linked compounds (silanes with structural unit of formula (III)), the signals for compounds of the respective formula (a) and (b) could be detected separately (i.e., the silanes with structural units of formula (IIa) and (IIb) could be quantified separately). In the region of the triple cross-links, the silanes could no longer be observed separately (i.e., a separation between silanes of formula (IVa) and (IVb) was no longer visible).

29Si-NMR

| | Measurement | Measurement | Measurement |
|---|---|---|---|
| | after 0 days | after 7 days | after 14 days |
| | Proportion | Proportion | Proportion |
| | Mol-% | Mol-% | Mol-% |
| Silanes of the formula (Ia) | 1.9 Mol-% | 3.4 Mol-% | 3.5 Mol-% |
| Silanes of the formula (Ib) | 5.7 Mol-% | 1.2 Mol-% | 0.9 Mol-% |
| Silanes with structural unit | 18.7 Mol-% | 20.9 Mol-% | 21.7 Mol-% |
| of the formula (IIa) | | | |
| Silanes with structural unit | 6.1 Mol-% | 6.8 Mol-% | 5.4 Mol-% |
| of the formula (IIb) | | | |
| Sliane with structural unit | 37.3 Mol-% | 30.1 Mol-% | 29.8 Mol-% |
| of the formula (IIIa) | | | |
| Silanes with structural unit | 0.5 Mol-% | 11.5 Mol-% | 13.6 Mol-% |
| of the formula (IIIb) | | | |
| Silanes with structural unit | 29.8 Mol-% | 26.1 Mol-% | 25.1 Mol-% |
| of the formula (IV) | | | |
| Total | 100 Mol-% | 100 Mol-% | 100 Mol-% |

### 3. Colorings

The following formulations were prepared (all data in wt. % unless otherwise stated):

Composition (B)

| | Gel |
|---|---|
| Hydroxyethylcellulose | 1.0 |
| Water (dist.) | ad 100 |

Composition (C)

| | in wt.. % |
|---|---|
| Lavanya Belmont | 35.0 |
| Phthalocyanine blue pigment CI 74160 | |
| PEG-12 Dimethicone | ad 100 |

Composition (D)

| | in wt.. % |
|---|---|
| Ethylene/Sodium Acrylate Copolymer (25% solution) | 40.0 |
| Water | ad 100 |

### 5. Application

For the dyeing tests, a silane blend (i.e., composition (A)) was used which was not stored (A-0), which was stored for a period of 7 days (A-7), and which was stored for a period of 14 days (A-14).

The ready-to-use composition was prepared by mixing 1.5 g of composition (A), 20.0 g of composition (B) and 1.5 g of composition (C), respectively. Compositions (A), (B) and (C) were each shaken for 1 minute, then this ready-to-use preparation was dyed on hair strands (Kerling, Euronatur hair white).

Three minutes after completion of shaking, the ready-to-use composition was applied to one strand at a time, left to act for 1 min, and then rinsed out.

Subsequently, the composition (D) was applied to each hair strand, left to act for 1 minute and then also rinsed with water.

The dyed strands were each dried and compared visually under a daylight lamp:

| Step 1: | (A-0) + (B) + (C) | (A-7) + (B) + (C) | (A-14) + (B) + (C) |
|---|---|---|---|
| Step 2: | (D) | (D) | (D) |
| Color intensity | 4 | 5 | 6 |

Color intensity: 1 = very low 6 = very high

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

What is claimed is:

1. Cosmetic composition for the treatment of keratinous material, said composition comprising:
(a) at least one silane of formula (I) 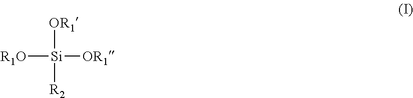
where
each of R1, R1′, R1″ independently is a hydrogen atom or a C₁-C₆ alkyl group,
R2 is a C₁-C₈ alkyl group or an amino-C₁-C₈ alkyl group, and
(b) at least one silane comprising at least one structural unit of formula (II), 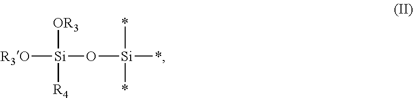
where
each of R3, R3′ independently is a hydrogen atom or a C₁-C₆ alkyl group, and
R4 is a C₁-C₈ alkyl group or an amino-C₁-C₈ alkyl group, and
(c) at least one silane comprising at least one structural unit of formula (III), 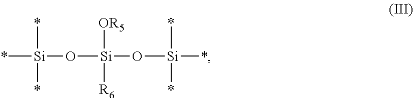
where
R5 is a hydrogen atom or a C₁-C₆ alkyl group, and
R6 is a C₁-C₈ alkyl group or an amino-C₁-C₈ alkyl group, and
(d) at least one silane comprising at least one structural unit of formula (IV), 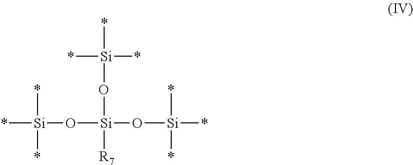
where
R7 is a C₁-C₈ alkyl group or an amino-C₁-C₈ alkyl group.

2. Composition according to claim 1, comprising at least one silane (a) of formula (Ia) 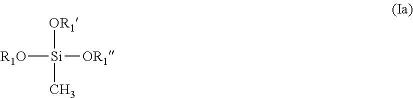
where
each of R1, R1′, R1″ independently is a hydrogen atom or a C₁-C₆ alkyl group.

3. Composition according to claim 1, comprising at least one silane (a) of formula (Ib) 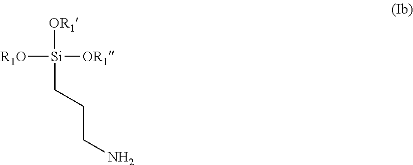
where
each of R1, R1′, R1″ independently is a hydrogen atom or a C₁-C₆ alkyl group.

4. A composition according to claim 1, comprising—based on the total molar amount of all silicon compounds used in the composition—(a) one or more silanes of formula (I) in a total mole fraction of from about 0.3 to about 12.0 mole %.

5. A composition according to claim 1, comprising—based on the total molar amount of all silicon compounds incorporated in the composition—(a) one or more silanes of formula (Ia) in a total mole fraction of from about 0.2 to about 7.0 mole %.

6. A composition according to claim 1, comprising—based on the total molar amount of all silicon compounds used in the composition—(a) one or more silanes of formula (Ib) in a total mole fraction of from about 0.1 to about 5.0 mole %.

7. Composition according to claim 1, comprising at least one silane (b) comprising at least one structural unit of formula (IIa), 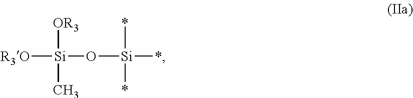
where
each of R3, R3′ independently is a hydrogen atom or a C₁-C₆ alkyl group.

8. Composition according to claim 1, comprising at least one silane (b) comprising at least one structural unit of formula (IIb), 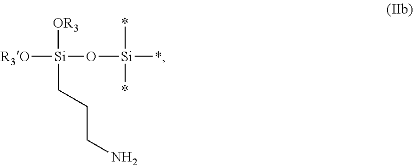
where
each of R3, R3′ independently is a hydrogen atom or a C1-C8 alkyl group.

9. A composition according to claim 1, comprising—based on the total molar amount of all silicon compounds used in the composition—one or more silanes (b) having a total molar content of from about 12.5 to about 42.0 mol % of structural units of the formula (II).

10. A composition according to claim 1, comprising—based on the total molar amount of all silicon compounds used in the composition—one or more silanes (b) having a total molar content of from about 12.0 to about 30.0 mol % of structural units of the formula (IIa).

11. A composition according to claim 1, comprising—based on the total molar amount of all silicon compounds used in the composition—one or more silanes (b) having a total molar content of from about 0.5 to about 12.0 mol % of structural units of the formula (Iib).

12. Composition according to claim 1, comprising at least one silane (c) comprising at least one structural unit of formula (IIIa), 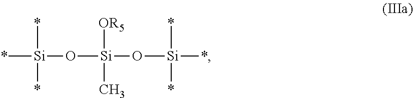
where
R5 is a hydrogen atom or a C₁-C₆ alkyl group.

13. Composition according to claim 1, comprising at least one silane (c) comprising at least one structural unit of formula (IIIb), 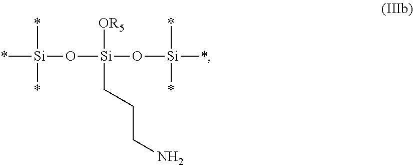
where
R5 is a hydrogen atom or a C₁-C₆ alkyl group.

14. A composition according to claim 1, comprising—based on the total molar amount of all silicon compounds used in the composition—one or more silanes (c) having a total molar content of from about 22.0 to about 60.0 mol % of structural units of the formula (III).

15. A composition according to claim 1, comprising—based on the total molar amount of all silicon compounds used in the composition—one or more silanes (c) having a total molar content of from about 16.0 to about 38.0 mol % of structural units of the formula (IIIa).

16. A composition according to claim 1, comprising—based on the total molar amount of all silicon compounds used in the composition—one or more silanes (c) having a total molar content of from about 6.0 to about 22.0 mol % of structural units of the formula (IIIb).

17. A composition according to claim 1, comprising at least one silane (d) comprising at least one structural unit of formula (IVa),

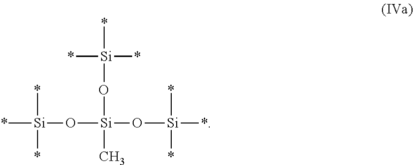

18. A composition according to claim 1, comprising at least one silane (d) comprising at least one structural unit of formula (IVb),

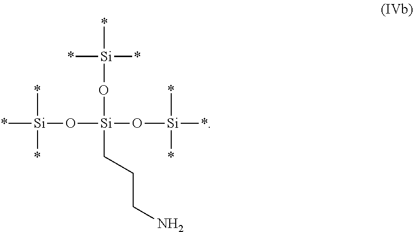

19. A composition according to claim 1, comprising—based on the total molar amount of all silicon compounds used in the composition—one or more silanes (d) having a total molar content of from about 18.0 to about 34.0 mol % of structural units of the formula (IV).

20. A composition according to claim 1, comprising, based on the total weight of the composition, one or more silanes (a) to (d) in a total amount of from about 30.0 to about 85.0% by weight.

21-24. (canceled)
